(19) United States
(12) Patent Application Publication
(10) Pub. No.: US 2024/0033345 A1
(43) Pub. Date: Feb. 1, 2024

(54) METHOD FOR PRODUCING VIRUS

(71) Applicants: Oxford University Innovation Limited, Oxford (GB); THE CHANCELLOR MASTERS AND SCHOLARS OF THE UNIVERSITY OF OXFORD, Oxford (GB)

(72) Inventors: Alexander DOUGLAS, Oxford (Oxfordshire) (GB); Carina Citra Dewi JOE, Oxford (Oxfordshire) (GB)

(21) Appl. No.: 18/039,833

(22) PCT Filed: Dec. 10, 2021
(86) PCT No.: PCT/EP2021/085242

§ 371 Date: Jun. 1, 2023

(30) Foreign Application Priority Data
Dec. 10, 2020 (GB) 2019455.1

(51) Int. Cl. A61K 39/215; C12N 15/86; C12N 7/00; C07K 14/165

(52) U.S. Cl. CPC A61K 39/215; C12N 15/86; C12N 7/00; C07K 14/165; C12N 2710/10351; A61K 2039/5256

(57) ABSTRACT

The invention relates to a method for preparing an adenovirus comprising a) providing a host cell in a medium capable of supporting growth of said host cell b) contacting said host cell with an adenovirus c) incubating to allow infection of said cell by said adenovirus d) incubating to allow production of adenovirus by said host cell wherein said host cell is, or is derived from, a HEK293 cell, and wherein the medium comprises BalanCD HEK293. The invention also relates to adenovirus produced, and to compositions comprising said adenovirus.

### FIELD OF THE INVENTION

The invention relates to a method of producing adenovirus, in particular adenovirus capable of expressing a heterologous antigen.

### BACKGROUND

Adenovirus vectored vaccines are among the leading approaches to vaccine development such as SARS-CoV-2 vaccine development. A perceived disadvantage, particularly in comparison to DNA and RNA vaccine platforms, is the complexity of adenovirus manufacturing. This typically involves a fed-batch or perfusion upstream process (USP), followed by a multi-step downstream process (DSP; most commonly depth filter clarification, tangential flow filtration [TFF], anion exchange chromatography [AEX], and a second TFF step) (Vellinga, J., et al., Challenges in manufacturing adenoviral vectors for global vaccine product deployment. Hum Gene Ther, 2014. 25(4): p. 318-27). The invention addresses problem(s) associated with large-scale process development and technology transfer to multiple manufacturing sites, seeking to improve upon the yield and scalability of previously reported processes (e.g. Fedosyuk et al 2019 Vaccine vol 37 pages 6951-6961—see below).

Methods of production of adenoviral vectors for use as vaccines and/or for other purposes (e.g. oncolytic, gene therapy) are known. High-yielding processes are valuable for the economical production of the vectors, including for commercial purposes, and for the rapid production of large quantities of vectors (for example in response to public health emergencies).

Numerous attempts have been made in the art to optimise yields of such production by o varying a wide variety of parameters (e.g. perfusion, temperature, oxygenation, cell density; Vellinga J, et al. Challenges in manufacturing adenoviral vectors for global vaccine product deployment. Hum Gene Ther. 2014; 25(4): 318-27.; Nadeau I, Kamen A. Production of adenovirus vector for gene therapy. Biotechnol Adv. 200 3; 20 (7-8): 475-89.; Galvez J, et al. Optimization of HEK-2935 cell cultures for the production of adenoviral vectors in bioreactors using on-line OUR measurements. J Biotechnol. 2012; 157(1): 214-22.; Shen C F et al. Optimization and scale-up of cell culture and purification processes for production of an adenovirus-vectored tuberculosis vaccine candidate. Vaccine. 2016; 34(29): 3381-7.)

It is important to note that in the art much focus has been made on using AdHu5 vectors. These replicate favourably in HEK293 cells and other cell lines expressing AdHu5 E1 gene products. However, these are sub-optimal for clinical use. It is also noted that many prior art methods use vectors carrying only model transgenes (e.g. green fluorescent protein [GFP], luciferase, or certain vaccine antigens which are known not to interfere with viral yields). It is much more challenging to make products based upon non-AdHu5 adenovirus serotypes and/or carrying diverse vaccine antigens, because many such antigens are known to interfere with adenovirus production yields and/or genetic stability. This is especially true for those from adenovirus species other than AdHu5′s species C (Douglas AD, et al. The blood-stage malaria antigen PfRH5 is susceptible to vaccine-inducible cross-strain neutralizing antibody. Nat Commun. 2011; 2: 601; Cottingham M G, et al. Preventing spontaneous genetic rearrangements in the transgene cassettes of adenovirus vectors. Biotechnol Bioeng. 2012; 109(3): 719-28.). Thus, efficient virus production remains a problem in the art.

To date the highest yielding processes known to the inventors are those using perfusion and the PER.C6 cell line (US20110207202A1). These processes use specialised equipment and methods which are challenging to transfer to new facilities, which are drawbacks. In addition, due to the process of continuous medium exchange, these processes consume a volume of medium which is larger than the bioreactor working volume, which is costly. Moreover, availability of PER.C6 cells is restricted since these are a proprietary cell line which is not universally available to the skilled worker.

Crucell's known perfusion-based process is disclosed in WO2011/098592, which describes a method for producing recombinant adenovirus serotype 26 (rAd26), the method comprising culturing producer cells in suspension with a perfusion system, and infecting said cells at a density of between 10e6 viable cells/mL and 16e6 viable cells/mL. This method involves an alternating tangential flow (ATF) perfusion system. This method is complicated and can be expensive. Also this process is challenging to transfer to manufacturing partners. Also this process achieves disadvantageous ratios of product to cells.

Typical yields of non-perfusion processes are in the region of 1e10 to 2e11 VP/mL. The inventors are not aware of any known non-perfusion processes achieving yields in excess of 2e11 VP/mL. Yields in non-perfusion processes are considered to be limited by a ‘cell density effect’ i.e. decreasing cell-specific yields (virus particles per cell) above a certain relatively low cell density, and then (above a higher cell density) falling volumetric yields. For this reason most authors reporting non-perfusion processes describe infection of cells at densities of c. 0.5-1e6 cells/mL, substantially below the cell densities which HEK293 or PER.C6 cells can reach in batch processes. Previously reported fed batch approaches have provided little benefit, leading to speculation that the cell-density effect is a result of accumulation of an unknown viral-replication-inhibitory factor, rather than depletion of a nutrient (Nadeau I, Kamen A. Production of adenovirus vector for gene therapy. Biotechnol Adv. 2003; 20(7-8): 475-89; Kamen A, Henry O. Development and optimization of an adenovirus production process. J Gene Med. 2004; 6 Suppl 1: S184-92).

Shen et al (Vaccine 2016 Jun. 17; 34(29): 3381-7) describes production of an AdHu5-based TB vaccine candidate, AdAg85A, by Cansino (China). Their best upstream productivity is ˜1e11 VP/mL (FIG. 2a, table 1 of Shen et al. It will be noted that the VP/mL titers in table 2 are describing titers after purification & concentration).

Maranga et al 2005 https://onlinelibrary.wiley.com/doi/epdf/10.1002/bit.20455 disclose strenuous efforts to enhance yields using a fed batch approach with PER.C6 cells. However, this essentially fails in terms of making more virus. The authors do not state a productivity in terms of VP/mL, but table III of Maranga et al shows it is not proportionate to cell density above 1e6 cells/mL, despite being successful in terms of maintaining the key cell nutrients.

The inventors assert that prior art attempts to produce adenovirus using fed batch processes were unsuccessful.

For example, Nadeau et al 2002 (Biotechnology and Bioengineering vol 77 pages 91-104) report yields in plaque forming units, for AdHu5-GFP, and the best result obtained is 1600 PFU/cell with a starting cell density of 1e6/ mL. Of course care must be taken in comparing yield in PFU/cell to yield in VP/mL but the inventor asserts that this is expected to be <<1e11 VP/mL. Nadeau et al 2002 also summarises other prior art attempts with problems of yield.

For example, Nadeau et al 1996 (Biotechnology and Bioengineering vol 51 pages 613-623) also discloses an example of fed batch for adenovirus production, but uses serum-containing medium, Ad5, and a complete medium exchange at the point of infection. This is cumbersome and/or not practical at large scale. Without this cumbersome step, the cell-specific productivity falls in this prior art teaching. Indeed the inventor asserts that Nadeau et al 1996 does not report the viral yield at all since the authors use the virus as a tool to express protein, and are focused on protein yield.

Thus the inventor concludes that in the literature it is accepted that fed batch does not achieve successful adenovirus production at useful yields.

Fedosyuk et al 2019 (Vaccine vol 37 pages 6951-6961 (doi: 10.1016)) discloses simian adenovirus vector production for early-phase clinical trials. A simple method applicable to multiple serotypes and using entirely disposable product-contact components is taught. This document addresses the problem of small or early-stage production. For example it is stated in the abstract “There is a need for rapid production platforms for small GMP batches of non-replicating adenovirus vectors for early-phase vaccine trials, particularly in preparation for response to emerging pathogen outbreaks.” The authors conclude that 2-4 Litre process size is comfortably adequate for their purposes. It is clear from Fedosyuk that the culture is a batch culture. The culture is described in Section 2.3 on page 6953 (left column) where it is stated “Vessels were seeded with 800 mL (Mobius®) or 1.2 L (BioBlu) of culture at 1.5-2.0×10⁶ cells/mL, then inoculated with virus at an MOI of 3. Three hours later, 1.5 volumes of fresh complete medium was added.” Thus there is no disclosure in Fedosyuk of a fed batch culture.

In more detail, Fedosyuk et al mentions just before section 2.4 “One-point calibration of the optical pH sensor was performed by taking a sample three hours after vessel set-up (allowing time for equilibration of the sensor, but before addition of the feed).” In Fedosyuk, the ‘feed’ is a 50:50 dilution of the cells in the ordinary growth medium at the time of infection, resulting in a low cell density (0.5-1e6/mL at the start of the viral growth period). This is not what would normally be understood as a ‘fed batch’ process—the Fedosyuk method is a batch process, with no further intervention after this dilution at the time of infection.

Thus, the prior art has a number of disadvantages. The present invention seeks to overcome problem(s) associated with the prior art.

### SUMMARY

The inventors were researching how to improve production and/or yields of adenovirus from culture systems. The inventors were surprised to discover that fed batch cultures could be manipulated to produce surprisingly improved yields compared to conventional cultures known in the art. The invention is based on these surprising findings.

In one aspect the invention relates to a method for preparing an adenovirus comprising
a) providing a host cell in a medium capable of supporting growth of said host cell
b) contacting said host cell with an adenovirus
c) incubating to allow infection of said cell by said adenovirus
d) incubating to allow production of adenovirus by said host cell

wherein said host cell is, or is derived from, a HEK293 cell, and

wherein the medium comprises BalanCD HEK293.

Suitably feed is added to said medium. Suitably the feed comprises BalanCD HEK293 Feed.

Suitably said host cell comprises a HEK293-T-REx cell.

Suitably said host cell comprises a EXPI293 inducible cell.

Suitably infection of said cell by said adenovirus is carried out at a cell density of at least 2e6 cells/mL.

Suitably feed is added to said medium in an amount of 5% of starting medium volume every 24-48 hours.

Suitably feed is added to said medium in the period 48 hrs before infection to 48 hrs after infection.

In one aspect the invention relates to a method for preparing an adenovirus comprising
a) providing a host cell in a medium capable of supporting growth of said host cell
b) contacting said host cell with an adenovirus
c) incubating to allow infection of said cell by said adenovirus
d) incubating to allow production of adenovirus by said host cell characterised by adding feed to said medium.

Suitably feed is added to said medium in an amount of 5% of starting medium volume at each addition.

Suitably feed is added to said medium every 24-48 hours. Suitably feed is added to said medium in an amount of 5% of starting medium volume every 24-48 hours.

Suitably feed is added to said medium for the period 48 hrs before infection to 48 hrs after infection.

Suitably the medium comprises BalanCD HEK293.

Suitably the feed comprises BalanCD HEK293 Feed.

Suitably the medium comprises BalanCD HEK293 and the feed comprises BalanCD HEK293 Feed.

Suitably feed is added to said medium in an amount of 5% of starting medium volume every 24-48 hours and feed is added to said medium for the period 48 hrs before infection to 48 hrs after infection.

Suitably the medium comprises BalanCD HEK293 and the feed comprises BalanCD HEK293 Feed and feed is added to said medium in an amount of 5% of starting medium volume every 24-48 hours.

Suitably the medium comprises BalanCD HEK293 and the feed comprises BalanCD HEK293 Feed and feed is added to said medium for the period 48 hrs before infection to 48 hrs after infection.

Suitably the medium comprises BalanCD HEK293 and the feed comprises BalanCD HEK293 Feed and feed is added to said medium in an amount of 5% of starting medium volume every 24-48 hours and feed is added to said medium for the period 48 hrs before infection to 48 hrs after infection. This is an especially suitable combination. For example, the feed comprises BalanCD HEK293 Feed and feed is added to said medium in an amount of about 5% of starting medium volume at about 0.5 h after infection and about 22 h after infection. Exemplary data are provided illustrating the excellent effects of this combination.

Suitably said host cell comprises a HEK293-T-REx cell.

It will be noted that simian Adenoviruses infect non-human primates (e.g. monkeys and/or chimpanzees) rather than humans. Each species contains a mix of human and simian (e.g. chimpanzee) viruses.

Suitably said adenovirus is, or is derived from, a simian adenovirus.

Suitably said adenovirus is, or is derived from, a species E adenovirus.

Suitably said adenovirus is, or is derived from, a species E simian adenovirus.

Suitably said adenovirus is, or is derived from, non-ChAd63 species E adenovirus.

Suitably the adenovirus is ChAdOx1 or ChAdOx2.

Suitably the adenovirus is ChAdOx1, or an adenovirus having capsid charge characteristics similar to, or substantially the same as, or preferably the same as, ChAdOx1.

More suitably the adenovirus is ChAdOx1.

Most suitably the adenovirus is ChAdOx1 nCoV-19.

Suitably said adenovirus comprises a nucleotide sequence capable of directing expression of Ad5 E4orf6 in said host cell.

Suitably said Ad5 E4orf6 comprises nucleotide sequence encoding the amino acid sequence of Uniprot Accession Number: Q6VGT3.

Suitably said Ads E4orf6 comprises the sequence of Uniprot Accession Number: Q6VGT3.

Ad5 E4orf6 sequence (Uniprot Q6VGT3):

```
MTTSGVPFGM TLRPTRSRLS RRTPYSRDRL PPFETETRAT
                50 60 70 80
ILEDHPLLPE CNTLTMHNVS YVRGLPCSVG FTLIQEWVVP
                90 100 110 120
WDMVLTREEL VILRKCMHVC LCCANIDIMT SMMIHGYESW
              130 140 150 160
ALHCHCSSPG SLQCIAGGQV LASWERMVVD GAMENQRFIW
              170 180 190 200
YREVVNYNMP KEVMEMSSVE MRGRHLIYLR LWYDGHVGSV
              210 220 230 240
VPAMSEGYSA LHCGILNNIV VLCCSYCADL SEIRVRCCAR
              250 260 270 280
RTRRLMLRAV RIIAEETTAM LYSCRTERRR QQFIRALLQH
              290
HRPILMHDYD STPM
```

Suitably said adenovirus comprises a heterologous nucleotide sequence capable of directing expression of an antigen of interest.

Suitably said antigen of interest comprises, or consists of, SARS-CoV2 spike protein.

Suitably said heterologous nucleotide sequence is under the control of the Tet Repressor.

Suitably the host cell expresses the Tet Repressor.

Suitably said host cell is contacted with said adenovirus at a multiplicity of infection (MOI) of 3 to 10.

Suitably said adenovirus is produced at a yield of at least 2×10¹⁴ vp/litre of medium.

In another embodiment the invention relates to a method for preparing an adenovirus wherein said method comprises use of a fed batch culture. The inventor does not know of any disclosure of a scalable (i.e. suspension cell) process for a simian or species E adenovirus. This illustrates the novelty of a fed batch production of species E and/or simian adenoviruses. Suitably the medium and/or feed conditions taught herein are included in such a fed batch process.

In another embodiment the invention relates to an adenovirus obtainable by a method as described above.

In another embodiment the invention relates to an adenovirus prepared by, or obtained by, a method as described above.

In another embodiment the invention relates to a composition comprising an adenovirus as described above.

Suitably said composition is a pharmaceutical composition.

Suitably said composition is a vaccine composition.

In an aspect, the invention provides a method for preparing an adenovirus comprising
a) providing a host cell in a medium capable of supporting growth of said host cell
b) contacting said host cell with a species E simian adenovirus, optionally ChAdOx1 nCoV-19,
c) incubating to allow infection of said cell by said adenovirus
d) incubating to allow production of adenovirus by said host cell

wherein said host cell is, or is derived from, a HEK293 cell, optionally a HEK293-T-REx cell,

wherein the medium comprises BalanCD HEK293, and

wherein the method comprises use of a fed batch culture, optionally wherein:
(i) feed is added to said medium in an amount of 5% of starting medium volume at each addition; and/or
(ii) feed is added to said medium within the period 48hrs before infection to 48 hrs after infection; and/or
(iii) the volume of liquid comprising host cells used to produce the adenovirus is at least 200 L; and/or
(iv) the cell-specific productivity is >100,000 VP per cell (sometimes >200,000 VP per cell) at cell densities exceeding 2e6 cells/mL.

In an aspect, the invention provides a method for preparing an adenovirus comprising
a) providing a host cell in a medium capable of supporting growth of said host cell
b) contacting said host cell with a species E simian adenovirus, optionally ChAdOx1 nCoV-19,
c) incubating to allow infection of said cell by said adenovirus
d) incubating to allow production of adenovirus by said host cell

wherein said host cell is, or is derived from, a HEK293 cell, optionally a HEK293-T-REx cell,

wherein the medium comprises BalanCD HEK293 catalogue number 91165 or 94137 from FUJIFILM Irvine Scientific,

wherein the method comprises use of a fed batch culture, wherein the feed medium is BalanCD HEK 293 Feed catalogue number 91166 from FUJIFILM Irvine Scientific, optionally wherein:
(i) feed is added to said medium in an amount of 5% of starting medium volume at each addition; and/or
(ii) feed is added to said medium within the period 48 hrs before infection to 48 hrs after infection; and/or
(iii) the volume of liquid comprising host cells used to produce the adenovirus is at least 200 L; and/or
(iv) the cell-specific productivity is >100,000 VP per cell (sometimes >200,000 VP per cell) at cell densities exceeding 2e6 cells/mL.

### DETAILED DESCRIPTION

The invention relates to an adenovirus vector manufacturing process. The process has been demonstrated at 3 litre (3 L) scale.

A cGMP-ready 200 L-scale (˜1 m dose) manufacturing process has subsequently been developed. This finds application in producing (e.g.) ChAdOx1 nCoV-19 vaccine at large scale.

ChAdOx1 and ChAdOx2 are viral vectors previously described (Morris S J S, et al. Simian adenoviruses as vaccine vectors. Future Virology. 2016; 11(9): 649-59; Dicks MD, et al. A novel chimpanzee adenovirus vector with low human seroprevalence: improved systems for vector derivation and comparative immunogenicity. PLoS One. 2012; 7(7): e40385.). Both are Species E simian adenoviruses, engineered to contain the AdHu5 E4orf6 coding sequence to improve manufacturing yield in AdHu5 E1-expressing producer cells. ChAdOx1 means the ChAdOx1 adenoviral vector as described in Dicks et al. (2012) PLoS ONE 7(7): e40385, and/or in WO2012/172277.

### ChAdOx1

ChAdOx1 is a replication-deficient simian adenoviral vector. Vaccine manufacturing may be achieved at small or large scale. Pre-existing antibodies to the vector in humans are very low, and the vaccines induce strong antibody and T cell responses after a single dose, whilst the lack of replication after immunisation results in an excellent safety profile in subjects of all ages. ChAdOx1 is described in Dicks M D J, Spencer A J, Edwards N J, Wadell G, Bojang K, et al. (2012) A Novel Chimpanzee Adenovirus Vector with Low Human Seroprevalence: Improved Systems for Vector Derivation and Comparative Immunogenicity. PLoS ONE 7(7): e40385, and in WO2012/172277. Both these documents are hereby incorporated herein by reference, in particular for the specific teachings of the ChAdOx1 vector, including its construction and manufacture.

For insertion of the nucleotide sequence encoding antigen (such as spike protein), suitably the E1 site may be used, suitably with the hCMV IE promoter. Suitably the short or the long version may be used; most suitably the long version as described in WO2008/122811, which is specifically incorporated herein by reference for the teaching of the promoters, particularly the long promoter.

It is also possible to insert antigens at the E3 site, or close to the inverted terminal repeat sequences, if desired.

In addition, a clone of ChAdOx1 containing GFP is deposited with the ECACC: a sample of E. coil strain SW1029 (a derivative of DH10B) containing bacterial artificial chromosomes (BACs) containing the cloned genome of AdChOx1 (pBACe3.6 AdChOx1 (E4 modified) TIPeGFP, cell line name “AdChOx1 (E4 modified) TIPeGFP”) was deposited by Isis Innovation Limited on 24 May 2012 with the European Collection of Cell Cultures (ECACC) at the Health Protection Agency Culture Collections, Health Protection Agency, Porton Down, Salisbury SP40JG, United Kingdom under the Budapest Treaty and designated by provisional accession no. 12052403. Isis Innovation Limited is the former name of the proprietor/applicant of this patent/application.

### ChAdOx2

This is a viral vector based on Chimpanzee adenovirus C68. The sequence is in the public domain as SEQ ID NO: 10 in GB patent application number 1610967.0.

In addition, a clone of ChAdOx2 containing GFP is deposited with the ECACC: deposit accession number 16061301 was deposited by Isis Innovation Limited on 13 Jun. 2016 with the European Collection of Cell Cultures (ECACC) at the Health Protection Agency Culture Collections, Health Protection Agency, Porton Down, Salisbury SP40JG, United Kingdom under the Budapest Treaty. Isis Innovation Limited is the former name of the proprietor/applicant of this patent/application.

### ChAdOx1 nCoV-19

ChAdOx1 nCoV-19 means the spike protein of nCoV-19 expressed from the ChAdOx1 vector. In more detail, “ChAdOx1 nCoV-19” means the ChAdOx1 adenoviral vector as described in Dicks et al. (2012) PLoS ONE 7(7): e40385, and/or in WO2012/172277, comprising a nucleotide sequence encoding a 32aa tPA leader fused to SARS-Cov-2 spike protein inserted at the E1 locus of the ChAdOx1 adenoviral vector under the control of the CMV (cytomegalovirus) ‘long’ promoter. In case any further detail is required “ChAdOx1 nCoV-19” means the spike protein of nCoV-19 expressed from the ChAdOx1 vector constructed as described in the art for example in van Doremalen et al 2020 “ChAdOx1 nCoV-19 vaccination prevents SARS-CoV-2 pneumonia in rhesus macaques” (van Doremalen et al 2020 Nature volume 586, pages 578-582) (bioRxiv preprint document id: https://doi.orghomm/2020.05.13.093195).

In one embodiment the invention relates to adenovirus prepared by a method as described above.

In one embodiment the invention relates to adenovirus obtainable by a method as described above.

For the avoidance of doubt, references to ‘upstream’ (US) methods are references to viral production methods before/whilst the virus remains in the culture vessel.

References to ‘downstream’ (DS) methods are references to viral purification methods from the point that the virus is removed from the culture vessel (e.g. harvesting/removing a quantity of culture comprising host cells/virus from the culture vessel).

The invention provides a highly efficient process for the manufacture of adenoviruses/adenoviral vectors, suitable for cGMP execution at a range of scales up to >10 m doses per batch. The invention has the further advantageously of a very low cost of goods.

The invention finds application in manufacture of adenovirus-vectored vaccines.

In one embodiment the invention provides a combination of cells, preferably HEK293-based, and medium/feed which enable upstream process yield of adenovirus >2e14 virus particles (VP) per L of culture (sometimes >1e15 VP/L).

It is an advantage that the method does not require perfusion/medium-exchange. Suitably the method does not comprise medium-exchange. Suitably medium-exchange is omitted from the method of the invention. Most suitably medium-exchange is specifically excluded from the invention.

Suitably the virus is an adenovirus, more suitably the virus is a species E adenovirus. In one embodiment the invention provides a combination of cells, preferably HEK293-based, and medium/feed which enable upstream process yield of adenovirus >4e14 virus particles (VP) per L of culture (sometimes >1e15 VP/L).

It is an advantage that the method does not require perfusion/medium-exchange.

It is an advantage that the method of the invention can produce >8000 doses per L from the culture, (before purification).

It is an advantage that the method of the invention can produce a cell-specific productivity of >100,000 VP per cell (sometimes >200,000 VP per cell) at cell densities exceeding 2e6 cells/mL.

15 It is an advantage that the method of the invention can deliver the cell-specific productivity above 200,000 VP per cell at cell densities at infection exceeding 4e6 cells/mL (i.e. high ratio of product to contaminants). It is an advantage that the method of the invention is, or is part of, a simplified, rapid & highly cost-effective harvest and purification process.

It is an advantage that the method of the invention can produce yield >2e15 VP per L of culture, using perfusion, with cell density <1e7 cells/mL.

Suitably the virus is an adenovirus carrying AdHu5 E4Orf6 substitutions.

Suitably the virus is ChAdOx1, ChAdOx2, or ChAd63.

Suitably the virus is ChAdOx1 nCoV-19.

Suitably an antigen expression-repressing host cell/antigen promoter combination is used.

Suitably a batch fed process is used.

Suitably the process is not a perfusion process.

In one embodiment the invention relates to a surprisingly effective combination of

1. A medium/feed strategy (most suitably Fujifilm BalanCD293 medium/feed) and/or:

2. Highly productive cell line (most suitably HEK293 T-REx cells—these deliver very good adenovirus production, even when the antigen expression is de-repressed) Optionally the combination further comprises:

3. Antigen-repressing cell/promoter combination (most suitably HEK293 T-REx cells and a Tet-repressing promoter controlling expression of the antigen of interest) Optionally the combination further comprises:

4. The adenovirus being produced carries AdHu5 E4orf6 (most suitably as carried by adenoviruses ChAdOx1, ChAdOx, ChAd63).

The invention finds application in production of adenovirus for vaccine manufacture and/or for other purposes e.g. oncolytic.

The invention finds application in manufacture of adenovirus-vectored vaccines, including for example a SARS-CoV-2 vaccine, for example a SARS-CoV-2 vaccine comprising ChAdOx1 nCoV-19.

Prior art Fedosyuk describes 2-4 Litre size (process size). The Fedosyuk process might be able to be executed at larger volumes—but suffers from problems of yield. Suitably the invention is carried out at sizes different to 2-4 Litres, more suitably larger than 2-4 Litres. Suitably the invention is carried out at >=200 L, which is commercially useful. Suitably the invention is carried out at 1000-2000 L.

Suitably the process size used is larger than 4 L. Suitably the process size is 10 L or more, suitably 50 L or more, suitably 200 L or more, suitably >200 L, suitably l000L or more, suitably 1000 L-2000 L, or even more. Suitably process size means volume of culture vessel e.g. volume of bioreactor or more suitably volume of liquid comprising host cells used to produce the adenovirus.

The invention finds application in manufacturing a chimpanzee adenovirus-vectored SARS-CoV-2 vaccine to meet global needs. Development of a high-yielding process for production of an adenovirus-vectored SARS-CoV-2 vaccine is described.

### Cell Medium

The cell medium (‘medium’) is suitably capable of supporting the host cell such as supporting maintenance or culture of the host cell. More suitably the medium is capable of supporting growth of the host cell. This should not be taken to imply that the host cells must always be in growth. As is apparent to the skilled worker, a host cell typically does not grow after infection with adenovirus. However, a host cell is typically growing at the point of infection with adenovirus. Therefore suitably the medium is capable of supporting that growth.

Suitably the medium is capable of supporting adenovirus productivity.

Suitably the medium is capable of supporting the growth of suspension-adapted HEK293 T-rex cells to >1e7 cells/mL.

Suitably the medium/feed combination is capable of supporting the growth of suspension-adapted HEK293 T-rex cells to >1e7 cells/mL.

Many cell media in the art contain natural components such as animal components which can lead to lot-to-lot variations.

Suitably the medium is animal component-free. This has the advantage of ensuring consistency.

Suitably the medium is a chemically defined medium. This has the advantage of ensuring consistency.

In one embodiment suitably the medium is BalanCD HEK293—Liquid (most suitably catalogue number 91165) from FUJIFILM Irvine Scientific.

In one embodiment suitably the medium is BalanCD HEK293—Powder (most suitably catalogue number 94137) from FUJIFILM Irvine Scientific. Suitably the powder is formulated to a liquid medium for use, in accordance with the manufacturer's instructions.

In one embodiment suitably the feed is—BalanCD HEK 293 Feed (most suitably catalogue number 91166) from FUJIFILM Irvine Scientific.

FUJIFILM Irvine Scientific address: Unit 31, Newtown Business Centre, Block D, Newtownmountkennedy, Co. Wicklow, Ireland.

Regarding Anti-Clumping Supplement (most suitably catalogue number 91150) from FUJIFILM Irvine Scientific, suitably this is not used in the method of the invention.

Suitably, the medium is supplemented with an anti-foam reagent (Emulsion C, Sigma). The amount added is variable and may be determined by the operator. Suitably the anti-foam reagent is added as required. Suitably the anti-foam reagent is added in just-sufficient quantities to suppress foaming. Most suitably anti-foam reagent is added for large-scale production.

BalanCD HEK 293 medium contains 6 g/L glucose and BalanCD HEK293 Feed contains 40 g/L glucose.

BalanCD HEK 293 contains 0.1% poloxamer.

BalanCD HEK 293 does not contain phenol red.

Reference to the medium/feed by supplier and catalogue number ensures reproducibility.

Certificates of Analysis are available from the supplier. These are available on a lot-by-lot basis, thereby ensuring reproducibility. These may be requested at http://www.irvinesci.com/industrial-cell-culture/icc-technical-resources/icc-certificates-of-analysis or by contacting the supplier at the address above.

The inventor asserts that the supplier would always change the name/number of the product if they changed the composition of the medium. This is standard practice in the industry.

Moreover, this product is made to fulfil regulatory requirements for bio-manufacturing, and the supplier states that a drug master file has been supplied to the FDA. (supplier statement: “Fulfills regulatory requirements: Chemically-defined, animal-component free; Drug Master File (DMF) filed with US FDA”.

Thus it is clear that any unexpected change in the media would risk significantly undermining regulatory & customer confidence in the reliability of prior understanding of drug manufacturing processes which use it & would be commercially untenable for the supplier. This is further evidence establishing the reliability of supply and reproducibility of the media.

Even if the supplier unexpectedly discontinued selling the media as an off-the-shelf product, they would still be able to provide it for custom orders. Custom medium compositions are commonplace, and the composition could simply be supplied to its current formulation as a custom order even if the supplier unexpectedly removed it from the ‘in stock’/‘off-the-shelf’ product range. Indeed, the supplier statement in their Frequently Asked Questions information for the BalanCD HEK 293 medium includes “. . . FUJIFILM Irvine Scientific will work with our customers to address their formulation inquiries on a case by case basis. Please contact us with your formulation requests.” (see http://www.irvinesci.com/industrial-cell-culture/icc-technical-resources/icc-faq/hek293-system-faq). This is further evidence establishing the expectation of consistent supply throughout the lifetime of the patent(s) which may be granted for this invention.

In any case the skilled person would have no difficulty in analysing the composition of the media described herein to determine their composition. This can be achieved using standard techniques—such as liquid or gas chromatography in combination with mass spectrometry—or using analysers—such as the Roche Cobas chemistry analyser or the Vitros 350 chemistry analyser. Such analysis would be routine for the skilled person.

Accordingly, the skilled person would be able to determine the composition of the culture media used herein, should they choose to.

Suitably the medium is used without any addition or dilution thereto (unless otherwise specified e.g. addition of feed as discussed herein.)

### Feed

In one embodiment suitably feed is added to the medium after infection of said cell by said adenovirus.

In one embodiment suitably feed is added to the medium before infection and after infection by said adenovirus.

In one embodiment suitably feed is added to said medium in the period 48 hrs before infection to 48 hrs after infection by said adenovirus.

Suitably the feed is used without any addition or dilution thereto (addition of feed to medium as taught herein is not considered ‘dilution’ since the feed itself is being used without dilution).

Suitably feed is added at a rate of 5% starting volume at each addition.

Suitably feed is added at a rate of 0.05 volumes at each addition.

Suitably feed is added at 0.5 h after infection.

Suitably feed is added at 22 h after infection.

Suitably feed is added within 24 hours of when cells reach a density of 1e6 (1×10⁶) cells/mL before infection.

Suitably feed is added when cells reach a density of 1e6 (1×10⁶) cells/mL before infection. Suitably feed is added when cells reach a density of 4e6 (1×10⁶) cells/mL before infection.

### Medium and Feed—Manufacturer's Instructions

The following is an excerpt taken from the instruction document “Product Sheet 41084_BalanCD_HEK293_Media_System_Rev3” published by the manufacturer FUJIFILM Irvine Scientific:

### BalanCD HEK293 System

| | | | Available Package | |
|---|---|---|---|---|
| Catalog # | Product | Format | Sizes** | |
| 91165 | BalanCD HEK293 | Liquid | 1 | L |
| 94137 | BalanCD HEK293 | Powder | 10 | L |
| 91166 | BalanCD HEK293 Feed | Liquid | 500 | mL |
| 91150 | Anti-Clumping Supplement | Liquid | 50 | mL |

**Additional package sizes are available upon request

### Intended Use

For research or further manufacturing use only.

### Product Description

The BalanCD HEK293 System is a chemically defined, animal component-free platform of media and supplement optimized for production of viral vectors and transient protein expression. The product system comprises of BalanCD HEK293 medium, BalanCD HEK293 Feed, and Anti-Clumping Supplement. BalanCD HEK293 System contains no hydrolysates, L-Glutamine, antibiotics, antimycotics, or any other undefined components, and is ready to use for suspension culture applications. BalanCD HEK293 medium contains 6 g/L glucose and BalanCD HEK293 Feed contains 40 g/L glucose. BalanCD HEK293 medium can be supplemented with BalanCD HEK293 Feed for high density cell culture, or with Anti-Clumping Supplement post-transfection to minimize cell aggregation.

### Quality Assurance

All quality control test results are reported on a lot specific Certificate of Analysis which is available at www.irvinesci.com or upon request.

### Storage Instructions and Stability

### Liquid Medium

Handle using aseptic techniques to avoid contamination. Store at 2-8° C. and protect from light. This product is stable for 12 months, when unopened and stored properly. Do not use after the assigned expiration date. Not validated for use beyond the unopened expiry shelf life. Do not use any bottle of medium that shows evidence of particulate matter or cloudiness.

### Powder Medium

Store dry at 2-8° C. protected from moisture in the atmosphere. This product is very hygroscopic and should be kept in a dry environment away from moisture. Bring the powder to room temperature before opening and re-seal tightly after use. The powder should be free flowing; do not use if it is caked. This product is stable for 24 months, when unopened and stored properly. Do not use after the assigned expiration date.

### Directions for Use

### Hydration Of Balancd HEK293 From Powder Medium

1. Add powder medium (21.32 g/L, Catalog #94137) to WFI (1000 mL/L, Catalog #9309 or equivalent) into an appropriately sized container.

2. Mix the solution approximately 10 minutes or until the powder is well dissolved (the solution may still appear cloudy at this point).

3. Add 2.20 g/L Sodium Bicarbonate to the solution and mix at moderate speed until completely dissolved.

4. Measure pH (expected range 6.7-7.4) and osmolality (expected range 280-320 mOsm/kg).

5. Sterile filter through a 0.2 μm filter membrane.

6. The solution can be stored in the dark at 2-8° C. for up to 1 year.

7. Supplement 20 mL/L of 200 mM L-Glutamine (Catalog #9317) to BalanCD HEK293 medium to reach 4 mM final concentration prior to use.

8. BalanCD HEK293 contains 0.1% poloxamer; however, an additional 0.05% to can be supplemented if necessary.

### Cell Recovery and Adaptation

1. Supplement BalanCD HEK293 medium (Catalog #91165 or 94137) with 4 mM L-Glutamine (Catalog #9317).

Aseptically transfer appropriate volume (30 mL) of supplemented medium into a baffled 125 mL shake flask and equilibrate in a 37° C., 5% CO₂ incubator.

2. Thaw frozen vial rapidly in a 37° C. water bath.

3. Transfer the cells to a culture flask with pre-equilibrated BalanCD HEK293 medium (from step 1) to achieve an initial cell density of 3×10⁵ cells/mL.

4. Incubate culture in a 37° C., 5% CO₂ incubator for 3 to 4 days.

5. Sub-culture cells following the Sub-culturing Procedure outlined below.
NOTE: Cells can be directly adapted into BalanCD HEK293. After a minimum of three passages in BalanCD HEK293, if cells have successfully adapted, viable cell density and percent viability should reach above 1.5×6 cells/mL, and 90%, respectively.

6. If severe cell aggregation is observed, continue passaging with the following recommendation:
a. Supplement 2 mL/L Anti-Clumping Supplement (Catalog #91150).

7. If cells grow slowly (less than 1×10⁶ cells/mL within 4 days) with viability below 90%, continue passaging with the following recommendations:
a. Increase seeding density to 0.5-1×10⁶ cells/mL.
b. Spin down and re-suspend cells into fresh medium at each passage.
c. Sequential adaption at ratios of 1:1, 1:2, 1:4, and 0:1 current medium to BalanCD HEK293.

### Sub-Culturing Procedure

1. Supplement BalanCD HEK293 medium (Catalog #91165 or 94137) with 4 mM of L-Glutamine (Catalog #9317).

2. Sub-culture cell stocks every 2 to 3 days to keep cells in early logarithmic growth phase. Seed at a density of 3-5×10⁵ cells/mL. Viable cell density and percent viability should reach above 1.5×10⁶ cells/mL and 90%, respectively within 3 days.
Note: It is strongly recommended to keep the cultures under 3×10⁶ cells/mL in order to achieve high transfection efficiencies.

3. If severe cell aggregation is observed, continue passaging with the following recommendations:
Maintain viable cell density below 2×10⁶ cells/mL.
If problem still persists, add 2 mL/L of Anti-Clumping Supplement (Catalog #91150).

Note: Anti-Clumping Supplement is designed for use at a dilution between 1:1000 (1 mL/L) and 1:100 (10 mL/L) depending on degree of clumping.

### Cryopreservation

1. Prepare required volume of freezing medium (90% cold BalanCD HEK293+10%

DMSO). Keep at 4° C. until ready to use.

2. Centrifuge appropriate number of healthy cells for 5 minutes at 200 g and decant or aspirate the supernatant without disturbing the cell pellet.

3. Re-suspend cells in cold freezing medium at a density of 1×10⁷ viable cells/mL (or other desired cell density based on user needs).

4. Aliquot 1 mL/vial (or desired volume) into sterile cryovials.

5. Gradually lower the temperature of the vials to −80° C. at a rate of −1° C./minute in an appropriate freezing container.

6. Once cells reach −80° C., transfer to liquid nitrogen vapor phase for long term storage.

### Feed and Supplement for BalanCD HEK293

BalanCD HEK293 medium can be supplemented with BalanCD HEK293 Feed and/or Anti-Clumping Supplement to support multiple applications utilizing HEK293 cells. Anti-Clumping Supplement may be added to cultures if cells start to aggregate. For cultures where this supplement is added, Anti-Clumping Supplement must be eliminated from the culture media l prior to transfection, as this supplement will completely inhibit transfection.

To remove Anti-Clumping Supplement from culture, spin down cells, then wash cells with either 1X PBS (Catalog #9240), or BalanCD HEK293 medium (catalog #91165). Resuspend cells in BalanCD HEK293 medium without Anti-Clumping

Supplement before proceeding with transfection.

| | BalanCD HEK293 | Anti-Clumping |
|---|---|---|
| | Feed (Catalog | Supplement |
| Purpose | #91166) | (Catalog #91150) |
| Cell stock | No* | Optional: add 1-2 mL/L, |
| sub-culturing | | if cells begin to aggregate |
| Post-transfection | Yes | Optional: add 1-2 mL/L |
| enhancement of | See section: | at least one day post |
| cell growth and | BalanCD HEK293 | transfection, if cells |
| protein yield | Feed Optimization | begin to aggregate |
| | Guideline | |
| Viral vector | No* | Optional: add 1-2 mL/L |
| production | | at least one day post |
| | | transfection, if cells |
| | | begin to aggregate |
| Enhancement of | Yes | Supplement 1 mL/L to |
| stably transfected | See section: | BalanCD HEK293 |
| cell growth and | BalanCD HEK293 | medium prior to use |
| recombinant protein | Feed Optimization | |
| yield | Guideline | |

*BalanCD HEK293 Feed is recommended when extending the culture duration for more than a week and is not recommended for use for cell stock sub-culturing or viral vector production.

### BalanCD HEK293 Feed Optimization Guideline

BalanCD HEK293 Feed can be evaluated with the suggested standard feed method shown below. However, optimization of feed schedule and volume is highly encouraged to achieve optimal culture performance.

### Expression by Transient Transfection

1. Growth Medium: BalanCD HEK293 with 4 mM L-Gln
2. Determine optimal feed volume. Evaluate total feed volume at a range of 12-20% as below.

% Feed volume = % of initial culture volume.

| | |
|---|---|
| Day Post Transfection | Total Feed |

| 1 | 2 | 3 | 4 | Volume |
|---|---|---|---|---|
| 3% | 3% | 3% | 3% | 12% |
| 4% | 4% | 4% | 4% | 16%* |
| 5% | 5% | 5% | 5% | 20% |

*Suggested standard feed method

3. Determine feed schedule using feed volume determined from above in step 2. Feeding can be split into 4 events at an equal volume. Example with 20% total volume shown.

Day Post Transfection

| 1 | 2 | 3 | 4 | 5 | 6 | 7 | 8 |
|---|---|---|---|---|---|---|---|
| 5% | 5% | 5% | 5% | | | | |
| | 5% | 5% | 5% | 5% | | | |
| | | 5% | 5% | 5% | 5% | | |
| | | | 5% | 5% | 5% | 5% | |
| 5% | | 5% | | 5% | | 5% | |
| | 5% | | 5% | | 5% | | 5% |

### Expression by Stable Transfection

1. Growth Medium: BalanCD HEK293 with 4 mM L-Gln and 1 mL/L Anti-Clumping supplement
2. Determine optimal feed volume: Evaluate total feed volume at a range of 10-25% as below.

% Feed volume = % of initial culture volume.

| Culture Day | | | | | Total Feed |
|---|---|---|---|---|---|
| 3 | 4 | 5 | 6 | 7 | Volume |
| 2% | 2% | 2% | 2% | 2% | 10% |
| 3% | 3% | 3% | 3% | 3% | 15% |
| 4% | 4% | 4% | 4% | 4% | 20%* |
| 5% | 5% | 5% | 5% | 5% | 25% |

*Suggested standard feed method

3. Determine feed schedule using feed volume determined from above in step 2. Feeding can be split into 5 events at an equal volume. Example with 20% total volume shown.

Culture Day

| 3 | 4 | 5 | 6 | 7 | 8 | 9 | 10 | 11 | 12 |
|---|---|---|---|---|---|---|---|---|---|
| 4% | 4% | 4% | 4% | 4% | | | | | |
| | 4% | 4% | 4% | 4% | 4% | | | | |
| | | 4% | 4% | 4% | 4% | 4% | | | |
| | | | 4% | 4% | 4% | 4% | 4% | | |
| 4% | | 4% | | 4% | | 4% | | 4% | |
| | 4% | | 4% | | 4% | | 4% | | 4% |

### Host Cell

Suitably the host cell is an in vitro host cell. Suitably the host cell is derived from a human embryonic kidney (HEK) cell. Suitably the host cell is derived from a human embryonic kidney (HEK) 293 cell. HEK293-derived cells may be obtained from any suitable source, and/or derived by the skilled worker.

Examples of HEK293-derived cells are provided herein. For example, HEK293 T-Rex cells are HEK293-derived cells. For example Expi293 cells are HEK293-derived cells. For example Expi293F cells are HEK293-derived cells (available from Thermo Fisher (Thermo Fisher Scientific, 168 Third Avenue, Waltham, MA 02451, USA) Catalogue number: A14527 or Catalogue number: A14528.). HEK293-derived cells also include Expi293F inducible cells (available from Thermo Fisher (Thermo Fisher Scientific, 168 Third Avenue, Waltham, MA 02451, USA) Catalogue number: A39241).

Suitably the host cell is an in vitro cell previously derived from a human embryonic kidney (HEK) cell; suitably the methods of the invention do not embrace the obtaining of cell(s) from the human or animal body; suitably the methods of the invention do not require the presence of the human or animal body.

Suitably the host cell is a human embryonic kidney (HEK) cell such as a HEK293-T-REx cell.

Suitably the host cell is a HEK293-T-REx cell such as catalogue number R71007 (also known as catalogue number R710-07) from Thermo Fisher (Thermo Fisher Scientific, 168 Third Avenue, Waltham, MA 02451, USA)

ThermoFisher describe the cells as: “T-RExTM Cells stably express the tetracycline repressor protein. They save significant time and effort when using the T-REx™ System. The T-REx™ Cell Lines are functionally tested by transient transfection with the positive control vector pcDNA™4/TOλacZ. T-REx™ Cell Lines exhibit extremely low basal expression levels in the repressed state and high expression upon induction with tetracycline.”

In case any further guidance is needed, we refer to the supplier's instructions (publication number MAN0000106 (Revision 5.0); Thermo Fisher (Thermo Fisher Scientific, 168 Third Avenue, Waltham, MA 02451, USA).

There are many subtypes of HEK293 cells. Many of them have previously been suspension adapted. The HEK293 T-REx subtype has been suspension adapted in the art (Fedosyuk et al 2019, Vaccine, vol 37 pages 6951-6961).

The choice of HEK293-T-REx cells is advantageous because these cells are commercially available (see above). Known techniques have relied on PERC6 cells which are proprietary and have restricted access. Therefore the person skilled in the art may not be able to practice known techniques due to being unable to access the required PERC6 cells. Thus it represents an advantage of the invention that the viral production is enabled using publicly available HEK293-T-REx cells.

As well as the fact that they are commercially available, the HEK293 T-REx cells have the advantage (as compared to other cells e.g. PER.C6) that they repress the expression of the vaccine antigen transgene, if the transgene expression cassette contains a so-called ‘tet repressible’ promoter (Stanton R J, Re-engineering adenovirus vector systems to enable high-throughput analyses of gene function. Biotechniques. 2008; 45(6): 659-62, 64-8). The inventors have found this to enhance adenovirus yields and/or genetic stability.

### E4orf6

In principle any E4orf6 from a simian adenovirus may be used. However it must be noted that for correct function the E4orf6 has to interact with the Ad5 E1 protein (expressed by the host cell) so the E4orf6 needs to be sufficiently similar to Ad5 E4orf6 to support this functional interaction with Ad5 E1.

Suitably the E4orf6 is Ad5 E4orf6. Ad5 means Human adenovirus C serotype 5 (HAdV-5) (Human adenovirus 5).

Suitably E4orf6 comprises the amino acid sequence of accession number Uniprot Q6VGT3 (E4orf6 protein sequence).

Suitably nucleotide sequence encoding E4orf6 corresponds to the E4orf6 coding sequence of accession number Genbank NC_001405.1 (whole adeno genome sequence encoding E4orf6) (E4orf6 coding sequence 33193 to 34077).

This has the advantage is that species E Chimp Ad E4orf6's fail to interact with the Ad5 protein.

### Database Release

Sequences deposited in databases can change over time. Suitably the current version of sequence database(s) are relied upon. Alternatively, the release in force at the date of filing is relied upon.

As the skilled person knows, the accession numbers may be version/dated accession numbers. The citeable accession numbers for the current database entry are the same as above, but omitting the decimal point and any subsequent digits.

GenBank is the NIH genetic sequence database, an annotated collection of all publicly available DNA sequences (National Center for Biotechnology Information, U.S. National Library of Medicine 8600 Rockville Pike, Bethesda MD, 20894 USA; Nucleic Acids Research, 2013 January; 41(D1): D36-42) and accession numbers provided relate to this unless otherwise apparent. Suitably the current release is relied upon. More suitably the release available at the effective filing date is relied upon. Most suitably the GenBank database release referred to is NCBI-GenBank Release 239: 15 Aug. 2020.

UniProt (Universal Protein Resource) is a comprehensive catalogue of information on proteins (‘UniProt: a hub for protein information’ Nucleic Acids Res. 43: D204-D212 (2015).). Suitably the current release is relied upon. More suitably the release available at the effective filing date is relied upon. Most suitably, the UniProt consortium European Bioinformatics Institute (EBI), SIB Swiss Institute of Bioinformatics and Protein Information Resource (PIR)'s UniProt Knowledgebase (UniProtKB) Release 2020_05 of 7 Oct. 2020 is relied upon.

### Repression/Expression

The Tet repressor system in the T-REx cells is as described by Yao et al, 1998 (Hum Gene Ther. vol 9(13): pages 1939-50 PMID: 9741432). This differs from both the tet-on/tet-off systems, in that the tet-repressor protein is in its native form rather than fused to another repression/activation domain and so binds to the tetO element in the promoter in the absence of tetracycline (and not in its presence), but the effect of this binding is to repress expression.

Suitably the promoter used to direct expression of an antigen of interest in the adenovirus being produced comprises the tetO element.

Optimally the method of the invention comprises repression in culture (e.g. in T-REx cells) and de-repression in wild-type cells (e.g. when administered to a subject). Suitably this is achieved by using neither tet nor dox.

Wild-type cells (e.g. a subject's cells when the adenovirus is administered to a subject such as a human subject) do not express the Tet-repressor protein. Therefore in use the expression of the heterologous nucleotide sequence directing expression of the antigen of interest (such as SARS2-CoV spike protein) is not repressed in the cells of the subject, and so efficient expression of said antigen is driven by the promoter (such as the CMV promoter) in the usual manner, resulting in stimulation of an immune response in the subject.

Suitably the medium does not comprise Tetracycline

Suitably the medium does not comprise Doxycycline

Suitably the feed does not comprise Tetracycline

Suitably the feed does not comprise Doxycycline

### Viral Particles (VP) vs Infectious Units (IU)

When comparing yields between AdHu5 processes and non-human-adenoviruses, it is important to note that doses are usually defined in terms of virus particles (VP), but yields are sometimes reported in terms of infectious units (IU). The conversion factor between these, the particle : infectivity ratio, is typically 5 to 20 for AdHu5, but 50 to 200 for a simian adenovirus. Even expressed in VP terms, human doses for AdHu5-based vaccines may be higher (e.g. 0.8-1.6e11 VP) (e.g. Zhu F C, et al. Safety and immunogenicity of a recombinant adenovirus type-5 vector-based Ebola vaccine in healthy adults in Sierra Leone: a single-centre, randomised, double-blind, placebo-controlled, phase 2 trial. Lancet. 2017;3 89(10069): 621-8.) than for most simian adenovirus-vectored vaccines (2.5-5e10 VP) (e.g. Morris S J S, et al Simian adenoviruses as vaccine vectors. Future Virology. 2016; 11(9)649-59); in IU terms, AdHu5 doses are thus much higher than non-human adenovirus doses. As a result, particular care needs to be taken in comparing results expressed in IU.

### Advantages

Technical differences and/or advantages are delivered by the invention compared to Fedosyuk et al 2019.

For example, Host Cells are suitably HEK293-T-REx—but the inventors assert that they may not have been used with fed batch in the art. The fact that the method enables use of cells which are an alternative to the proprietary PERC6 cells is beneficial.

For example, Vector is suitably Species E simian Ad with Ad5 E4orf6 but the inventors assert that they may not have been used with fed batch in the art.

For example, suitably Antigen repression is used—but the inventors assert that may not have been used with fed batch in the art.

For example, in the art the medium/feed is [Batch: CD293, no feed] whereas in the method of the invention suitably the medium/feed is [Fed batch: BalanCD293 medium], [with feed]. This technical change compared to prior art methods leads to the benefit of yield improvement. This is applicable to at least ChAdOx1 & ChAdOx2 adenoviral vectors. The inventors assert that this is generally applicable to other species E adenoviruses/adenoviral vectors. The inventors assert that this may be generally applicable to all adenoviruses/adenoviral vectors.

The Fedosyuk method is a batch process, with no further intervention after this dilution at the time of infection. In the process of the invention, the feed differs from the growth medium (the feed is a much more concentrated nutrient solution) and is added in small volumes, with little diluting effect. In one embodiment the inventor defines a fed batch as the addition of feed after the infection has happened.

Multiplicity of infection is suitably 3-10.

Viral yields are improved compared to known methods. Suitably the methods of the invention deliver viral yields of 2e14 vp/litre (2e11 vp/mL). The inventors assert that these yields are higher than any known method. The inventors assert that these yields may be up to wo% higher than yields from known methods (i.e. the inventors assert that these yields may be up to 2× yields from known methods.)

Methods of the invention are simpler compared to known methods.

The invention provides improved viral production with simplified methods.

Methods of the invention provide improved ratio of product to cell-derived contaminants. This advantageously also provides the technical benefit of improving the efficiency of downstream steps (e.g. purification steps) such as making filtration easier.

Methods of the invention substantially increase the amount of adenovirus produced per litre of culture. Methods of the invention make it easier to purify the virus (e.g. virus for vaccine) because there is a high ratio of product to cells at the time of harvest (start of purification).

The invention delivers a beneficial technical effect on adenovirus manufacturing yield, genetic stability and product-to-product consistency via the use of cell line/promoter combinations which repress the expression of the heterologous antigen from the virus during manufacture.

Methods of the invention substantially increase the amount of adenovirus produced per litre of culture.

Methods of the invention make it easier to purify the virus (e.g. to manufacture a vaccine). Methods of the invention provides a high ratio of product to cells at the time of harvest (start of purification).

The process makes it possible to obtain ˜4000 doses of vaccine per L of culture. A known process such as a perfusion-based process may achieve a similar level but is more complicated & expensive & harder to transfer to manufacturing partners, and does not achieve such favourable ratios of product to cells. Thus the inventor asserts that the process of the invention obtains 4×-20× the yields of known methods such as:

˜200 doses per litre (Vaccitech/Advent)

˜1000 doses per litre (Tianjin Cansino)

The process of the invention makes it possible to obtain ˜8000 doses of vaccine per L of culture in a cost-effective and simplified manner.

Thus the inventor asserts that the process of the invention obtains 8×-40× the yields of known methods such as:

˜200 doses per litre (Vaccitech/Advent)

˜1000 doses per litre (Tianjin Cansino)

This provides benefits such as making the economics of virus/viral vector production significantly more favourable. This also makes it possible to prepare larger quantities of virus/viral vector more rapidly, which is important in an emergency situation (e.g. a pandemic of a life-threatening virus) and/or if first to market with such a product.

The inventors assert that this is the first fed batch Adenovirus production process to achieve these levels of productivity.

The inventors assert that this is the first Adenovirus production method/process to achieve these levels of productivity without perfusion.

The inventors assert that this is the first fed batch Adenovirus production method/process with such productivity.

The inventors assert that this is the first Adenovirus production method/process to permit a two-unit-operation downstream process.

A 2-operation downstream could be achieved with a low cell density, high cell specific productivity but low volumetric productivity process.

Compared to known methods such as Maranga et al 2005 https://onfinelibrary.wiley.com/doi/epdf/10.1002/bit.20455 (see background section—with PER.C6 cells.), we show clearly that yield is proportionate to cell density up to 2e6 cells/mL for ChAdOx1nCoV, and higher for some other viruses. This is an advantage of the invention.

In one embodiment suitably cell density >=2e6 cells/mL at point of infection.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### BRIEF DESCRIPTION OF THE DRAWINGS

Embodiments of the present invention will now be described further, with reference to the accompanying drawings, in which:

FIG. 1 shows graphs, bar charts and a table.

FIG. 2 shows graphs, diagrams and tables.

FIG. 3 shows graphs, tables and a diagram.

FIGS. 4A and 4B show graphs of medium adaptation.

FIGS. 5A and 5B show graphs of growth and feeding.

FIG. 6A and 6B show a bar chart and a table of yield in shake flasks (ChAdOx1 luciferase)(Experiment CJ7).

FIG. 7 shows a bar chart of yield in shake flasks (ChAdOx2 GFP) (Experiment CJ9)

FIG. 8 shows a plot of yield in shake flasks (ChAdOx1 Lassa) (Experiment CJ9)

FIG. 9 shows a bar chart of volumetric productivity in different culture media.

FIG. 10 shows graphs of volumetric productivity and cell-specific productivity at different cell densities at infection.

### EXAMPLES

All results in Examples 1-10 were obtained with BalanCD293 medium/feed combination & HEK293 T-REx cells.

ChAdOx1 nCoV-19 manufacturing scale up: 50 L and 200 L runs were performed at Pall Biotech, Portsmouth, UK.

### Example 1: Growth Of ChAdOx1 Luciferase in Shake Flasks

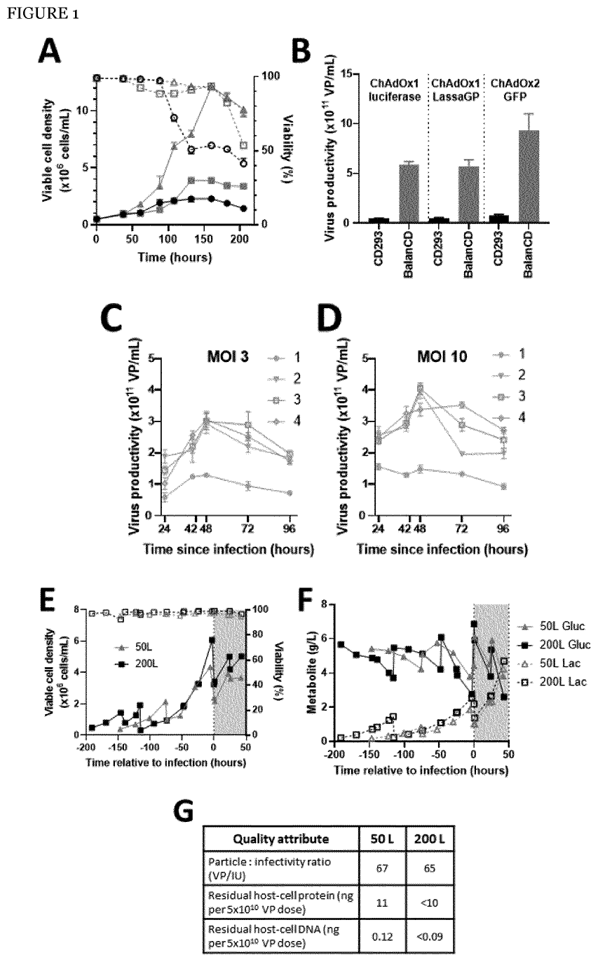

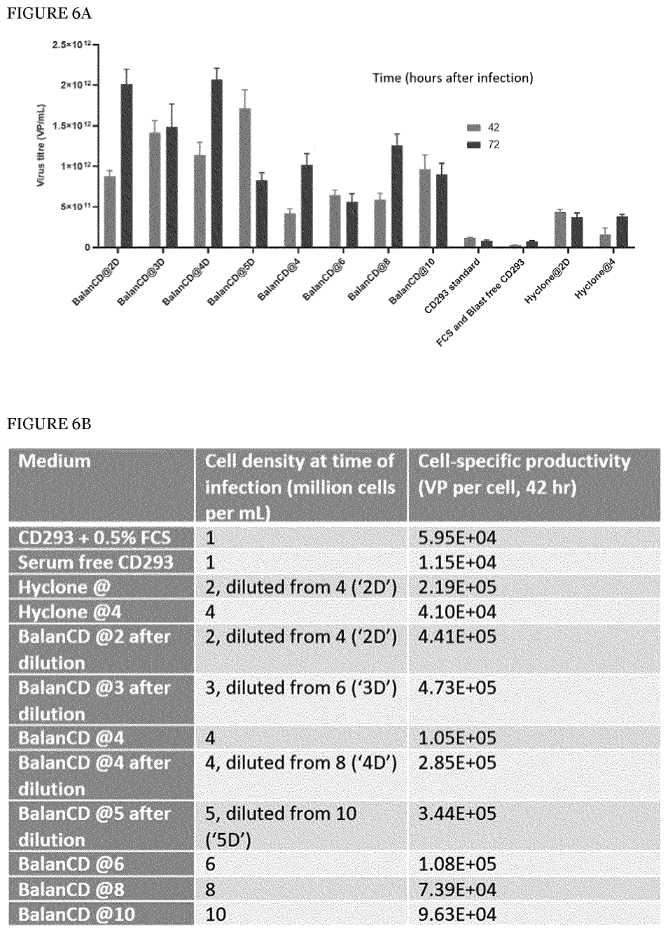

We refer to FIG. 6. We refer FIG. 1A.

‘D’ indicates use of the medium/feed strategy stated in Methods, including 2-fold dilution immediately prior to infection. Lack of ‘D’ indicates the strategy was modified by the omission of this 2-fold dilution.

### Example 2: Growth of ChAdOx2 GFP in Shake Flasks

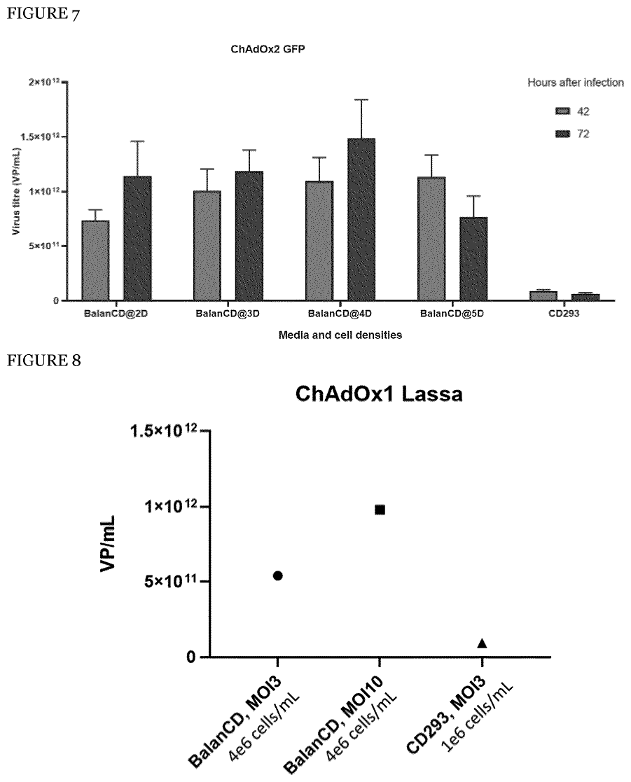

We refer to FIG. 7. We refer to FIG. 1B.

### Example 3: Growth of ChAdOx1 LassaSpike in Shake Flasks

We refer to FIG. 8. We refer to FIG. 1B.

### Example 4: Growth of ChAdOx1 Luciferase in 3L Stirred Tank Bioreactor

A 3L stirred tank reactor (STR) was infected and harvested as previously described (Fedosyuk et a1 2019 Vaccine vol 37 pages 6951-6961), with the exception of the use of the BalanCD293 medium/feed strategy, with a starting cell density of 4e6 cells/mL (diluted from 8e6 cells/mL) and an MOI of 10. Yield is shown in the Table below:

TABLE
ChAdOx1 luciferase upstream process
yield from 3L STR (experiment CJ10)

| | VP/mL | IU/mL | P:I |
|---|---|---|---|
| BalanCD luciferase bioreactor 1 | 5.8E+11 | 1.4E+10 | 41 |

### Example 5: 50 L Stirred Tank Bioreactor Production & Purification of ChAdOx1 nCoV-19

A 40 L culture was infected and harvested in a 50 L STR, as previously described (Fedosyuk et al 2019 Vaccine vol 37 pages 6951-6961 (doi: 10.1016)), with the exception of the use of the BalanCD293 medium/feed strategy, with a starting cell density of 2e6 cells/mL (diluted from 4e6 cells/mL) and an MOI of 10.

The product was subsequently purified using a strategy as previously described (Fedosyuk et a1 2019 Vaccine vol 37 pages 6951-6961 (doi: 10.1016)). The final purified product yield was 1.8×10¹¹ VP per mL of the upstream process (FIG. 1E-G).

### Example 6: Comparative Data

Here we illustrate the improvement (e.g. in yield) compared to known methods. Comparative data is presented.

The inventor asserts that the benchmark in the art is yields <1e11 VP/mL for non-perfusion processes.

Firstly, specific to simian Adenoviruses, we refer to FIG. 1B.

This shows side-by-side comparisons for 2× ChAdOx1 & 1× ChAdOx2 viruses.

This clearly shows the prior art process performs at a much lower productivity, and the methods of the invention deliver a clear technical benefit compared to the prior art process.

In addition, we refer to Table 1 in Fedosyuk et al 2019. This shows productivity of the known process across 8 runs with 3 viruses. The highest is 1.4e11 VP/mL and the mean is <1e11 VP/mL (=1e14 VP/L).

### Example 7: Production/Upstream Process

We initially investigated medium/feed combinations for a fed-batch USP. BalanCD293 medium and feed (Fujifilm) was found to support growth of the producer cells (HEK293 T-rex, Thermo) to 1.2×10⁷ cells/mL with high viability (FIG. 1A). Using this combination in small-scale production of adenovirus vectors of two serotypes and carrying three transgenes, we attained productivity exceeding 5×10¹¹ virus particles (VP) per mL, around 5-fold greater than typically obtained in our previous USP (FIG. 1B). To our knowledge such productivity has not previously been reported from a non-perfusion USP: a fall in cell-specific productivity to <1×10⁵ VP/cell is commonly observed at cell densities exceeding 1×10⁶ cells/mL (Kamen, A. and O. Henry, Development and optimization of an adenovirus production process. J Gene Med, 2004. 6 Suppl 1: p. S184-92).

Using ChAdOx1 nCoV-19 starting material, we initially investigated the optimal multiplicity of infection (MOI), cell density and time of harvest (FIG. 1C-D). An MOI of 10, cell density of 2-3×10⁶/mL and time of harvest 42-48 hours post infection were selected for further work. This USP was subsequently scaled up, with USP productivities in 10 L, 50 L and 200 L stirred tank reactors (STR) in the range 2-4×10¹¹ VP/mL, and acceptable cell growth and metabolite profiles (FIGS. 1E-F).

Early STR batches made use of a DSP very similar to that we had previously described, which again achieved recovery of 50-60% and quality characteristics compliant with a regulator-accepted specification for product for clinical use (FIG. 1G) (Fedosyuk et al 2019 Vaccine vol 37 pages 6951-6961).

We refer to FIG. 1 which shows development of fed batch process.

Panel A shows cell counts (solid lines, filled symbols) and viability (dashed lines, open symbols) attained during growth of HEK293 T-rex cells in BalanCD medium with (triangles) or without (squares) feed, as compared to the CD293 medium (circles, Thermo) used in our previous process. In the case of the BalanCD medium +feed condition, feed (5% v/v) was added at 36 and 108 hours.

Panel B shows small-scale USP productivity of ChAdOx1-luciferase, ChAdOx1-LassaGP, and ChAdOx2-GFP with BalanCD medium/feed (infected at 4×10⁶ cells/mL, MOI=10) as compared to our previously established conditions in CD293 medium (infected at 1×10⁶ cells/mL, MOI=3) (Fedosyuk et al 2019 Vaccine vol 37 pages 6951-6961).

ChAdOx1-luciferase infections were performed in a 3L bioreactor; the other two viruses were produced in 30 mL volume in shake flasks.

Panels C-D show small-scale USP productivity of ChAdOx1 nCoV-19 in shake flasks at 30 mL working volume at MOI=3 (C) and MOI=10 (D) respectively. Legend indicates cell density represented by each line, in million cells/mL at point of infection.

Infectious unit (IU) titers broadly paralleled VP titers; results are representative of two replicate experiments.

For panels A-D, points indicate median and error bars show range of results for 2-3 replicate flasks.

Panels E-F: Examples of 50 L and 200 L USP runs (carried out by Pall Biotech). (E) shows cell growth (solid lines) and viability (dashed lines). (F) shows glucose (solid lines) and lactate (dashed lines) concentrations.

Panel G: Examples of quality of drug substance from 50 L and 200 L runs (carried out by Cobra).

### Example 8: Purification/Downstream Process

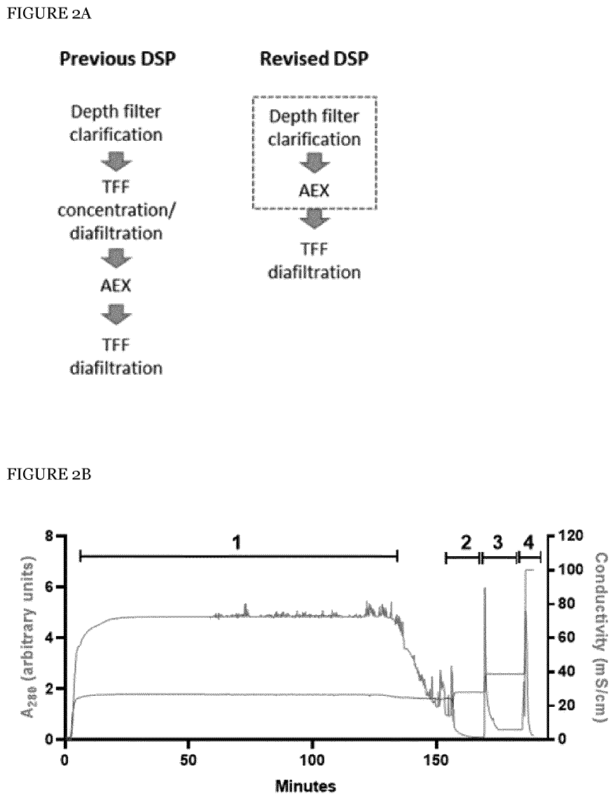

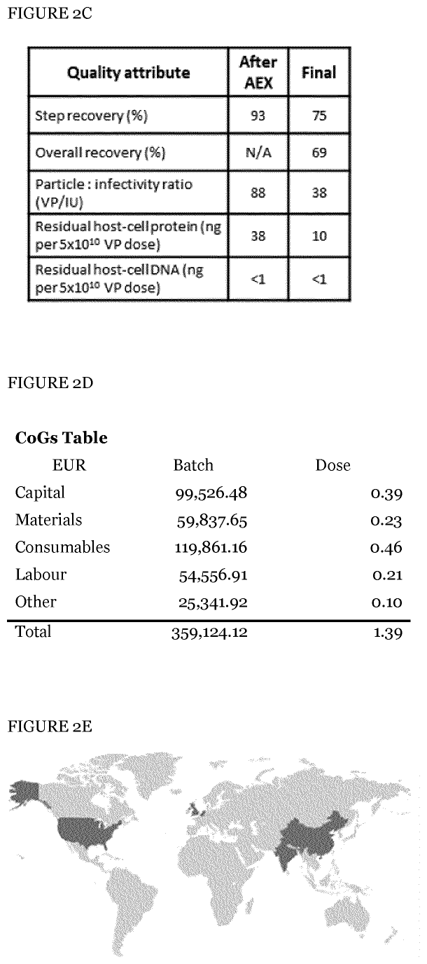

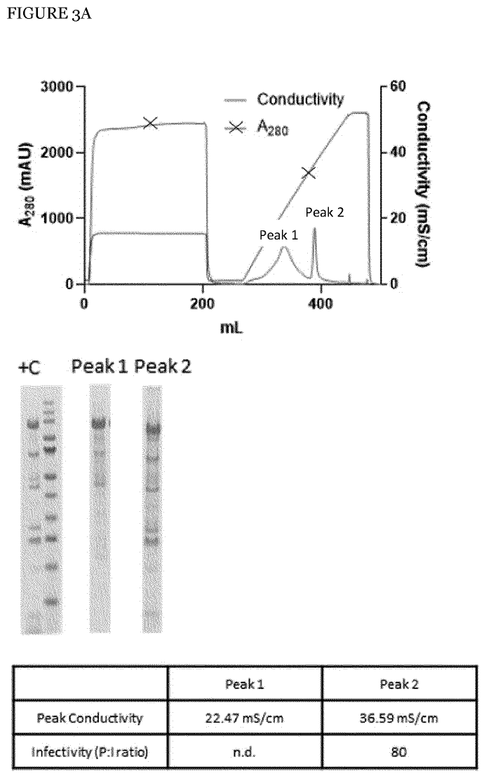

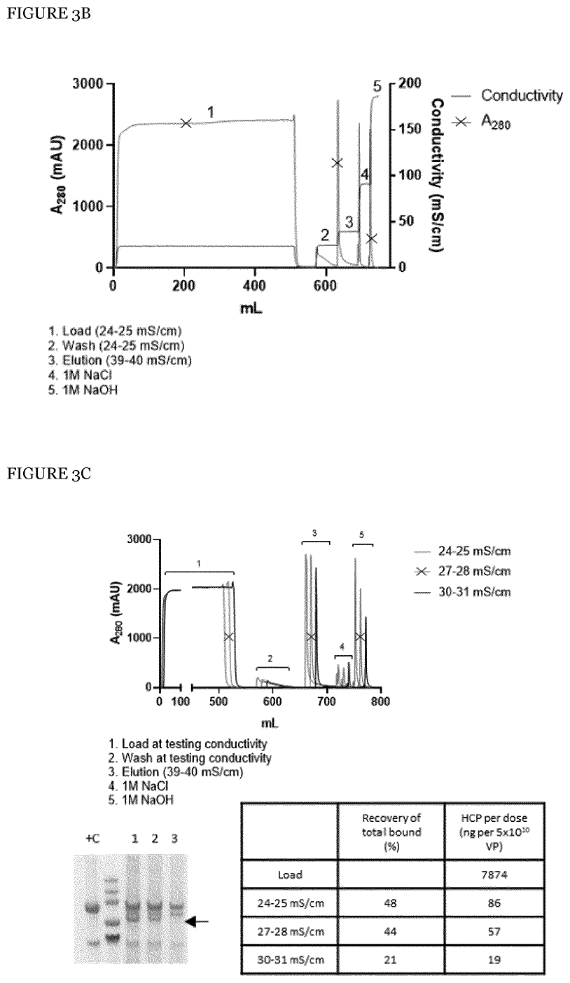

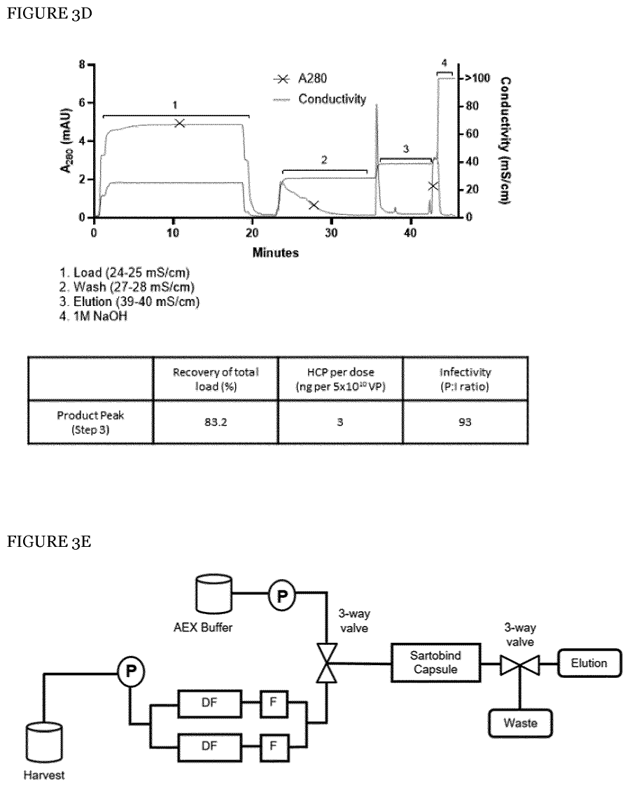

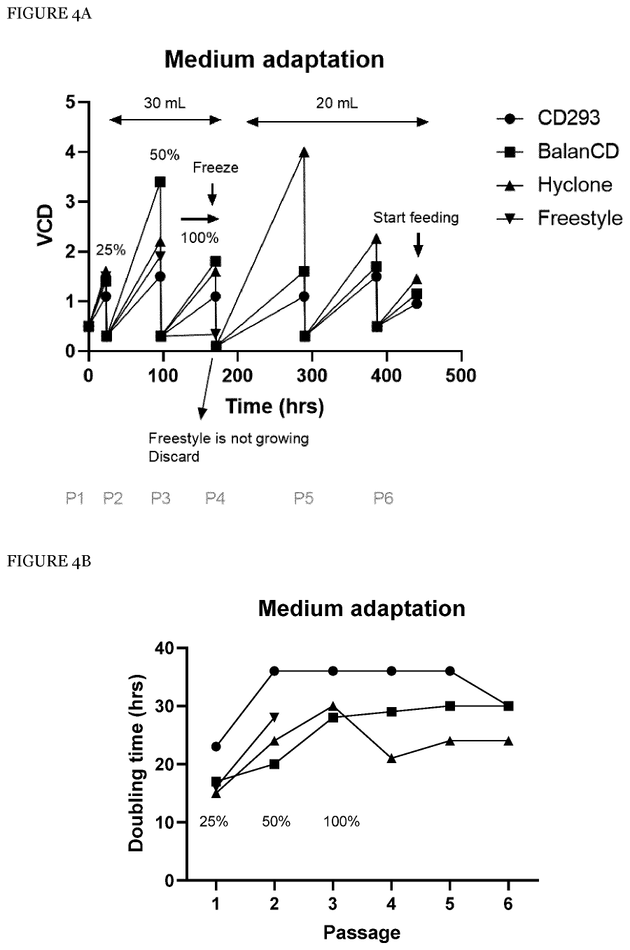

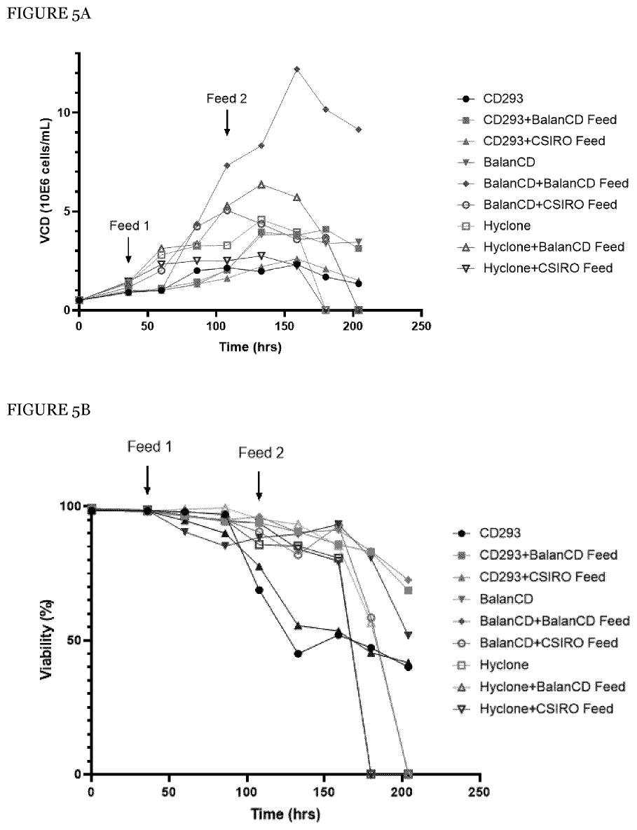

The inventors proceeded to scale the process to 1000-2000 L. Handling of the large volumes of lysate, diafiltration buffer and waste during the initial TFF step was identified as a process bottleneck. We therefore developed a simplified DSP by loading the clarified lysate directly on an AEX membrane, followed by a single TFF diafiltration polish/formulation step. As well as removing the initial TFF step, this process change provides the option of execution of clarification and AEX as a single unit operation (FIG. 2A). Small-scale experiments were used to demonstrate feasibility and optimise conditions of the AEX step (FIG. 3A-D), prior to execution of the complete revised DSP at 10 L scale (FIG. 2B). Binding capacity of the AEX membrane was reduced (to c. 3×10¹³ VP per mL of membrane, as compared to >5×10¹³ VP with loading of a diafiltered feed), but it was typically possible to recover c. 90% of loaded virus in the eluate. After a final TFF step, overall recovery was typically c. 70% and host cell protein and DNA were reduced to acceptable levels (FIG. 2C). Efficient recovery of adenovirus from such a ‘direct load’ AEX has not, to the inventors' knowledge, previously been disclosed. The ability to achieve this enabled the simplification of the DSP from four steps to two or three.

The simplicity and high productivity of the method of the invention are beneficial from an economic perspective. Process economic modelling of drug substance manufacturing suggests a cost of goods of <USD 2 per dose (FIG. 2D). Final prices may be somewhat higher due to additional costs, for example fill and finish.

### Example 9: Distributed Manufacturing

COVID-19 poses a unique challenge for vaccine manufacturing, both due to the volume and speed required, and due to concerns about equitable vaccine distribution and so-called ‘vaccine nationalism’. ‘Distributed manufacturing’ (manufacturing the same vaccine across multiple facilities in different countries) is a potential solution to these issues, but requires a readily transferable process (Gomez, P. L. and J. M. Robinson, Vaccine Manufacturing. Plotkin's Vaccines, 2018: p. 51-60.e1). Our process is well-suited to this strategy as it uses unit operations which are standard across the bioprocess industry, single-use product-contact materials throughout, and a viral vector of good biosafety (BSL1-2, dependent upon jurisdiction). Many contract manufacturing organisations (CMOs) are already equipped to execute such processes with little or no capital expenditure. We therefore pursued a distributed manufacturing strategy from an early stage of the pandemic. ChAdOx1 nCoV-19/AZD1222 drug substance is now being manufactured at 1000 L scale in facilities in multiple countries (FIG. 2E).

We refer to FIG. 2 which shows Improved process, enabling cost-effective large-scale distributed manufacture

Panel A shows schematics of previous and revised DSPs. The dashed box indicates the feasibility of execution of depth filter clarification and AEX as a single unit operation. Panel B shows chromatogram obtained when running ‘in-line’ depth filter clarification and AEX at 10 L scale. Absorbance at 280 nm is shown (line showing 4-5 arbitrary units between 50 and 100 minutes), and conductivity in also shown (other line). For schematic of process skid, see FIG. 3E. A 150 mL Sartobind Q capsule was loaded at 3.8×10¹³ VP per mL of membrane. Numerals indicate stages: 1=loading, 2=wash, 3=elution, 4=1M sodium hydroxide sanitisation.

Panel C shows product recovery and quality from the 10 L process shown in Panel B and after final formulation by TFF.

Panel D tabulates modelled costs of drug substance production at 200 L scale. This includes capital for purchase of all equipment needed for the process; many facilities do not require this.

Panel E shows, in dark grey, countries in which ChAdOx1 nCoV-19/AZD1222 is currently being manufactured.

We refer to FIG. 3 which shows small-scale optimisation of anion exchange with direct loading of clarified lysate.

Panel A shows gradient elution of ChAdOx1-luciferase from Sartobind Q anion exchange membrane. Filtered lysate containing 9×10¹³ VP of ChAdOx1-luciferase was loaded onto a 3 mL Sartobind nano Q capsule, followed by elution with a gradient of increasing salt concentration. Two peaks were observed (chromatogram) and analysed. Coomassie-stained SDS-PAGE, with comparison to virus purified by caesium chloride gradient ultracentrifugation (+C), shows that peak 1 contains impurities (notably free hexon protein) while Peak 2 contains virus. This was corroborated by infectivity assay. “n.d.” indicates “not detected”.

Panel B shows initial estimation of binding capacity and product recovery by step elution. Clarified lysate containing 2×10¹⁴ VP of ChAdOx1 nCoV-19 was loaded onto a 3 mL Sartobind nano Q capsule. Binding capacity and quantity of product bound was determined by collection of serial fractions of flow-through during loading (step 1). After washing with equilibration buffer (step 2), product was eluted with a step to 39-40 mS/cm (step 3); steps 4 and 5 indicate regeneration with 1M NaCl and 1M NaOH respectively. Flowthrough and elution fractions were analysed by qPCR to calculate binding capacity and recovery: 10% breakthrough occurred at a load of 3.4×10¹³ VP per mL of bed volume; 90% of bound product was recovered.

Panel C shows optimisation of salt concentration/conductivity during loading and washing. Three runs were performed, in each of which filtered lysate containing 2×10¹⁴ VP of ChAdOx1 nCoV-19 was loaded onto a 3 mL Sartobind nano Q capsule, after adjustment of conductivity to the indicated values by addition of salt. Each run used wash buffer with conductivity matching the load. The chromatogram overlays results from the three runs. Eluates were analysed by Coomassie-stained SDS-PAGE, qPCR and HCP ELISA. Recovery and HCP are tabulated: the ‘basal’ condition (24-25 mS/cm) achieved nearly 2-log₁₀ reduction in HCP; increasing the conductivity of the load and wash achieved modest improvement in HCP clearance, with substantially reduced recovery with loading at 30-31 mS/cm. As virus in the flowthrough was not quantified, recovery is shown as % of loaded product (rather than bound product, as in panel B); the low recovery for the basal condition here thus reflects the overloading of the column.

Panel D shows initial results obtained using a 150 mL Sartobind Q capsule with relatively low load challenge. Clarified lysate containing 7.4×10¹⁴ VP of ChAdOx1 nCoV-19 was loaded, using conditions as indicated, based upon the results of the experiment shown in Panel C. The eluate was analysed by qPCR, HCP ELISA and infectivity assay. Recovery as % of loaded product, HCP and P:I ratio are tabulated.

Panel E illustrates system used for in-line clarification and anion exchange (P: pump; DF: depth filter; F: 0.2 μm filter), results of which are shown in FIG. 2B-C.

### Viruses

The ChAdOx1 nCoV-19, ChAdOx1 Lassa-GP, ChAdOx1 luciferase and ChAdOx2 GFP vectors used here have previously been described¹⁻⁴. Virus used as seed to infect shake flask cultures and as standards in quality control assays was produced by caesium chloride density-gradient ultracentrifugation by the Jenner Institute Viral Vector Core Facility. Virus used as seed to infect 50 L and 200 L cultures was prepared using our previously described process in 3 L shake flasks or bioreactors, up to the point of the first tangential flow filtration (TFF) step⁵. After this the concentrated and diafiltered lysate was aliquoted and frozen at −80° C.

### Cells and Upstream Process

HEK293 T-rex cells (ThermoFisher) were banked and adapted to low-serum suspension culture in CD293 medium (ThermoFisher) as previously described⁵. Cells were then adapted to increasing proportions of BalanCD293 medium (Fujifilm-Irvine Scientific), supplemented with 4 mM GlutaMAX (ThermoFisher), over one week.

For upstream process experiments, seed culture at 2× the specified final density was diluted by addition of 1 volume of fresh medium to reach the cell density specified for each experiment at the point of infection. A multiplicity of infection of 10 was used unless otherwise stated.

Each feed was with 0.05 volumes of BalanCD293 feed (Fujifilm-Irvine Scientific). Pre-infection, cultures were fed on the day cell density exceeded 1×10⁶ cells/mL. Cultures for which the intended cell density at the point of infection was 3×10⁶ cells/mL received a second pre-infection feed when cell density exceeded 4×10⁶ cells/mL. Post-infection, all cultures were fed at 0.5 h and 22 h after infection.

Shake flask experiments were performed in Erlenmeyer flasks (Corning), with a working volume of 25-35 mL in a 125 mL flask unless otherwise stated. BioBlu 3c and 14c (Eppendorf) single-use bioreactor vessels were used in accordance with the manufacturers' instructions. A GX bioreactor controller unit and C-BIO software (both from Global Process Control) were used to control both vessel types. Dissolved oxygen (DO) was regulated at a setpoint of 55% air saturation by addition of medical air via macrosparger. pH was regulated in the range 7.2-7.3 as previously described⁵.

50 L and 200 L upstream processes were performed using Pall Allegro stirred tank reactors (STRs).

Unless otherwise stated, bioreactors were seeded at 0.4-0.6×10⁶ cells/mL in c. 35% of the maximum working volume. Antifoam C emulsion (SigmaAldrich) was used in 50 L and 200 L STRs. 0.05 culture volumes of BalanCD feed was added when the density reached to 1.0×10⁶ culture cells/mL. At a cell density of 4.0×10⁶/mL (range 3.0-6.0×10⁶), cells were diluted with 1 volume of medium and infected, using an MOI of 10 unless otherwise stated. 0.05 volumes of BalanCD feed were added 30 minutes after infection, and again after 22 hours.

### Lysis, Nucleic Acid Digestion and Clarification

Lysis was performed as previously described⁵, in the culture vessel, with the exception that the concentration of Benzonase (MerckMillipore) was reduced to 15 units/mL. Lysis was initiated at 42-48 h after infection, with the exception of the productivity kinetic experiments shown in FIGS. 1C-D. Two hours after addition of lysis buffer, clarification was initiated, using Millistak+® HC Pro CoSP depth filters as in our previous work⁵. During 200 L runs, an Advanced MVP skid (Pall Biotech) was used for filtration steps.

### Tangential Flow And Bioburden Reduction Filtration

Tangential flow filtration was performed essentially as we have previously described⁵, scaled appropriately and with the following modifications. Where TFF was performed before anion exchange (AEX), i.e. for the 200 L run producing product as reported in FIG. 1G, only 2-fold concentration was performed, prior to 6 diavolumes of diafiltration. For TFF after AEX, Omega T-series 300 kDa cut-off flat sheet filters (Pall Biotech) were used. For TFF during 200 L runs, an Allegro CS 4500 single-use TFF skid was used (Sartorius). A Supor EKV 0.2 μm filter was used for bioburden reduction filtration after the final TFF.

### Anion Exchange Chromatography

Where preceded by TFF (run reported in FIG. 1G), AEX was performed as previously reported⁵, with scaling of the chromatography capsule and buffer volumes based upon anticipated binding capacity of 7×10¹³ VP per mL of membrane volume.

For ‘direct-load’ AEX (loading clarified lysate), small-scale studies (Supplementary FIG. 1) were performed using an Akta Pure (GE) and 3 mL bed volume/8 mm bed height SartobindQ Nano capsules (Sartorius). Equilibration buffer comprised 20 mM Tris-HCL pH8.0, 1 mM MgCl₂, 0.1% v/v polysorbate 20, 5% w/v sucrose; this was also used as a base for wash buffers. Elution buffer comprised 20 mM Tris-HCL pH8.0, 1 mM MgCl₂, 0.1% v/v polysorbate 20, 5% w/v sucrose, 600 mM NaCl, except where salt concentration was varied, as stated. Adjustment of the conductivity of the sample and wash buffers, to target values as stated in the descriptions of individual experiments, was performed using 5M NaCl (Sigma).

Column equilibration was in accordance with the manufacturer's instructions. After loading, capsule was washed with 10 MV of equilibration buffer before elution step (both at 5 MV/min).

For the ‘direct-load’ AEX purification from a 10 L bioreactor (FIG. 2B) a peristaltic pump-driven rig was constructed, as shown in Supplementary FIG. 1E, incorporating a CoSP depth filter (as above), Millipak-20 0.2 μm filter, and 150 mL/8 mm bed height Sartobind Q capsule (Sartorius), plus single-use UV absorbance, conductivity and pressure sensors (Pendotech). Buffers, column equilibration, sample loading, washing and elution were as described above, with the exception that a flow rate of 0.7 membrane volumes/minute was used for sample loading, washing and elution.

### Product Quantification and Assessment Of Product Quality

Product quantification was as previously reported, using qPCR and UV spectrophotometry assays for viral particles in impure and pure samples respectively, and an immunostaining-based infectivity assay⁵.

Residual host-cell protein (HCP) was quantified using the HEK293 HCP ELISA kit (Cygnus Technologies), according to the manufacturer's instructions. Residual host cell DNA was quantified using a previously reported quantitative PCR method targeting a 94 base pair amplicon within the Alu repeats⁶. The lower limit of quantification was 100 pg/mL for intact HEK293 DNA.

### References to Examples 7, 8 and 9

1 van Doremalen, N. et al. ChAdOx1 nCoV-19 vaccine prevents SARS-CoV-2 pneumonia in rhesus macaques. Nature, doi: 10.1038/s41586-020-2608-y (2020).

2 Purushotham, J., Lambe, T. & Gilbert, S. C. Vaccine platforms for the prevention of Lassa fever. Immunol Lett 215, 1-11, doi: 10.1016/j.imlet.2019.03.008 (2019).

3 Dicks, M. D. et al. Differential immunogenicity between HAdV-5 and chimpanzee adenovirus vector ChAdOx1 is independent of fiber and penton RGD loop sequences in mice. Sci Rep 5, 16756, doi: 10.1038/srep16756 (2015).

4 Morris, S. J. S., Sarah; Spencer, Alexandra J.; Gilbert, Sarah C. Simian adenoviruses as vaccine vectors. Future Virology 11, 649-659, doi: 10.2217/fvl-2016-0070 (2016).

5 Fedosyuk, S. et al. Simian adenovirus vector production for early-phase clinical trials: A simple method applicable to multiple serotypes and using entirely disposable product-contact components. Vaccine, doi: 10.1016/j.vaccine.2019.04.056 (2019).

6 Zhang, W. et al. Development and qualification of a high sensitivity, high throughput Q-PCR assay for quantitation of residual host cell DNA in purification process intermediate and drug substance samples. J Pharm Biomed Anal 100, 145-149, doi: 10.1016/j.jpba.2014.07.037 (2014).

### Example 10: Antigen Repression

Here we show data which illustrate that the antigen repression, such as provided by the most suitable HEK293 T-REx cells, delivers the advantage of high productivity (enhanced productivity compared to non-repression of antigen).

The conditions used were the BalanCD293 fed batch production process (‘upstream process’) as described above.

The data show that for the most suitable specific product (ChAdOx1 nCoV-19) there is a strong enhancement of productivity with repression compared to without repression.

The data also show that this is not an essential feature of the invention (i.e. repression is not always necessary for production of all adenoviruses), but is rather an advantageous embodiment.

Thus it is shown that the benefit of the method of the invention (fed batch upstream process) is indeed generalisable to other cell lines (i.e. to cells other than the most suitable HEK293 T-REx cells).

Suitably when the adenovirus comprises nucleic acid encoding an antigen capable of being expressed which is inhibitory to the adenovirus production, expression of said antigen is repressed.

### Results

1. Chadox1-nCov19 in T-REx

a. with tetracycline (which removes the repression, allowing antigen expression):

4.3E+09 VP/mL

b. without tetracycline (i.e. with the antigen repressed): 2.4E+11 VP/mL

2. In Expi293 cells (i.e. no tet repression)

a. Chadox1-ncov19 (antigen expressed): 1.1E+10 VP/mL

b. Chadox1-luciferase (antigen expressed but the luciferase protein is relatively non-interfering for adeno production): 1.9E+11 VP/mL

### Example 11: Production in EXPI293 Inducible Cells

Here we show data that illustrate that the use of BalanCD media with the HEK293-derived cells, Expi293F inducible cells (available from Thermo Fisher (Thermo Fisher Scientific, 168 Third Avenue, Waltham, MA 02451, USA) Catalogue number: A39241) achieves a more than 1.5 fold increase in volumetric productivity compared with the same cells grown in the Expi293 medium. This increased volumetric productivity is demonstrated for both culture in a shaker, and culture in a stirred tank reactor.

### Methods

For both the experiments with Expi293F-inducible cells in BalanCD medium (in shaker and stirred tank reactor), frozen cells were revived in 30 mL volume of Expi293 medium. 48 hours later cells were split 1:2 in the same volume with 15 ml of fresh medium. 24 hours later, cell density was adjusted to 0.5e6 cells/ml final viable cell density (VCD) with 75% volume (22.5 mL) Expi293 medium and 25% (7.5 mL) BalanCD medium. 24 hours later further adapted the cell by adjusting the density to 1 e6 cells/ml final VCD with 50% volume (15 mL) Expi293 medium and 50% (15 mL) BalanCD medium. 48 hours later, adjust density to 0.5e6 cells/ml final VCD with BalanCD medium. Thereafter cells were diluted with 100% BalanCD medium.

For the experiments in the shaker:

Expi293F inducible cells were cultured in 125 mL shake flasks in duplicates. 0.05 culture volumes (i.e. 5% of the starting medium volume) of BalanCD feed was added when the density reached 1e6 culture cells/mL. At a cell density of 4.4e6 cells/ml, cells were diluted with 1 volume of medium (final volume of 30 mL) and infected, using an MOI of 5. Subsequently, 0.05 volumes of BalanCD feed were added 30 minutes after infection, and again after 22 hours. The cultures were then harvested 47 hour after infection. At harvest 1/9 volume of 10× Lysis buffer and 100 IU/ml benzonase was added into each flask and incubated for 2 hours before they were diluted 1:200 in A438 formulation buffer for qPCR or non-diluted for infectivity analysis. All samples were frozen at −80° C. before analysis.

For the experiments in the reactor:

3 L upstream processes were performed using 3cBioBlu Eppendorf stirred tank reactors (STRs). The process was run as above, with the exception that dissolved oxygen was maintained at 55% and pH was adjusted with 7.5% sodium bicarbonate. The cultures were then harvested 47 hour after infection, as above.

### Results

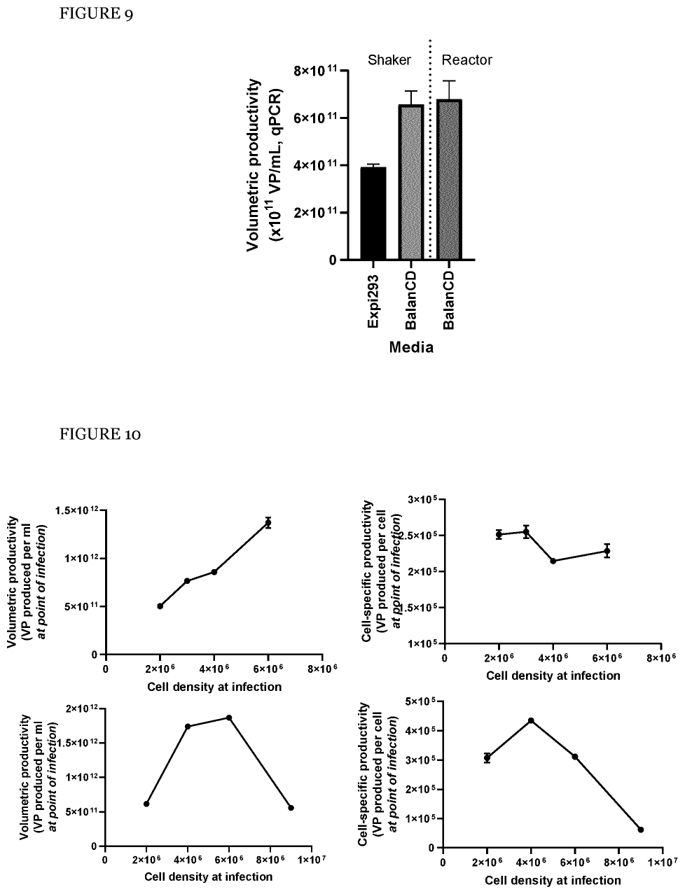

FIG. 9, left and middle columns show the comparison of volumetric productivity of ChAdOx1-nCoV19 in a shaker using Expi293F inducible cells in Expi293 medium (left column) and BalanCD medium (middle column). This shows a more than 1.5 fold increase in volumetric productivity when using BalanCD medium compared to Expi293 medium.

FIG. 9, right column shows the volumetric productivity of ChAdOx1-nCoV19 in a reactor (3c Bioblu) using Expi293F inducible cells in a 3L scale using BalanCD medium. This demonstrates that the increased volumetric productivity resulting from the use of BalanCD medium is maintained when using a different batch fed systems.

### Conclusion

These data clearly demonstrate that the use of BalanCD media in a fed batch system results in an increased volumetric yield across different HEK293-derived cell types, and that this increase is consistent across different fed batch systems.

### Example 12

This Example shows that the use of BalanCD medium and feed with Expi293 inducible cells allows at least maintenance of cell-specific productivity at cell densities exceeding 2×10⁶ cells/mL at point of infection. BalanCD medium therefore permits higher volumetric productivities than have previously been reported for adenovirus production using fed batch processes.

### Methods

Two similar experiments were performed to assess volumetric productivity and cell-specific productivity at different cell densities at the point of infection.

In the first experiment (corresponding to top two panels of FIG. 10) cells were cultured in 125 mL shake flasks in duplicates for each of the cell densities, 0.05 culture volumes of BalanCD feed was added when the density reached 1.0 and 4.0×10⁶ culture cells/mL. The cells were then infected at different densities (2.0, 3.0, 4.0, 6.0×10⁶ cells/ml), using an MOI of 5. Subsequently, 0.05 volumes of BalanCD feed were added 30 minutes after infection, and again after 22 hours. The cultures were then harvested 47 hour after infection. At harvest 1/9 volume of 10× Lysis buffer and 100 IU/ml benzonase was added into each flask and incubated for 2 hours before they were diluted 1:200 in A438 formulation buffer for qPCR. All samples were frozen at −80° C. before analysis.

In the second experiment (corresponding to bottom two panels of FIG. 10) cells were cultured in 125 mL shake flasks in duplicates for each of the cell densities, 0.05 culture volumes of BalanCD feed was added when the density reached 1.0 and 5.0×10⁶ cells/mL. The cells were then infected at different densities (2.0, 4.0, 6.0, 9.0×10⁶ cells/ml), using an MOI of 5. Subsequently, 0.05 volumes of BalanCD feed were added 30 minutes after infection, and again after 22 hours. The cultures were then harvested and samples prepared for qPCR as above.

Both experiments were performed with ChAdOx1-nCoV19.

### Results

Both experiments demonstrate that the cell-specific productivity can be maintained at cell densities exceeding 2×10⁶ cells/mL at point of infection. This therefore allows a higher volumetric productivity than methods which involve use of a cell density of equal to or less than 2×10⁶ cells/mL at point of infection. Both of these experiments demonstrated that the highest volumetric productivity is achieved with a cell density at infection of 6.0×10⁶ cells/ml

Although illustrative embodiments of the invention have been disclosed in detail herein, with reference to the accompanying drawings, it is understood that the invention is not limited to these precise embodiments and that various changes and modifications can be effected therein by one skilled in the art without departing from the scope of the invention as defined by the appended claims and their equivalents.

1. A method for preparing an adenovirus comprising
a) providing a host cell in a medium capable of supporting growth of said host cell
b) contacting said host cell with an adenovirus
c) incubating to allow infection of said cell by said adenovirus
d) incubating to allow production of adenovirus by said host cell
wherein said host cell is, or is derived from, a HEK293 cell, and
wherein the medium comprises BalanCD HEK293

2. A method according to claim 1 wherein feed is added to said medium, and wherein the feed comprises BalanCD HEK293 Feed.

3. A method according to any preceding claim wherein said host cell comprises a HEK293-T-REx cell, an Expi293F cell, or an Expi293F inducible cell.

4. A method according to any preceding claim wherein infection of said cell by said adenovirus is carried out at a cell density of at least 2e6 cells/mL, optionally wherein infection of said cell by said adenovirus is carried out at a cell density of between 4e6 cells/mL to 7e6 cells/mL, preferably about 6e6 cells/mL.

5. A method according to any preceding claim wherein feed is added to said medium in an amount of 5% of starting medium volume every 24-48 hours.

6. A method according to any preceding claim wherein feed is added to said medium in the period 48 hrs before infection to 48 hrs after infection.

7. A method according to any preceding claim wherein said adenovirus is, or is derived from, a simian adenovirus.

8. A method according to any preceding claim wherein said adenovirus is, or is derived from, a species E simian adenovirus.

9. A method according to any preceding claim wherein the adenovirus is ChAdOx1 or ChAdOx2.

10. A method according to claim 9 wherein the adenovirus is ChAdOx1.

11. A method according to claim 10 wherein the adenovirus is ChAdOx1 nCoV-19.

12. A method according to any preceding claim wherein said adenovirus comprises a nucleotide sequence capable of directing expression of Ad5 E4orf6 in said host cell.

13. A method according to claim 12 wherein said Ad5 E4orf6 comprises the sequence of Uniprot Accession Number: Q6VGT3.

14. A method according to any preceding claim wherein said adenovirus comprises a heterologous nucleotide sequence capable of directing expression of an antigen of interest.

15. A method according to any preceding claim wherein said antigen of interest comprises, or consists of, SARS-CoV2 spike protein.

16. A method according to any preceding claim wherein said heterologous nucleotide sequence is under the control of the Tet Repressor.

17. A method according to any preceding claim wherein the host cell expresses the Tet Repressor.

18. A method according to any preceding claim wherein said host cell is contacted with said adenovirus at a multiplicity of infection (MOI) of 3 to 10.

19. A method according to any preceding claim wherein said adenovirus is produced at a yield of at least 2×10¹⁴ vp/litre of medium.

20. A method according to any preceding claim wherein said method comprises use of a fed batch culture.

21. A method for preparing an adenovirus wherein said method comprises use of a fed batch culture.

22. An adenovirus prepared by a method according to any preceding claim.

23. A composition comprising an adenovirus according to claim 22.

24. A composition according to claim 23 which is a pharmaceutical composition.

25. A composition according to claim 24 which is a vaccine composition.


SEQUENCE LISTING (1 sequence)

SEQ ID NO: 1 - protein, 294 aa, Human adenovirus type 5
MTTSGVPFGMTLRPTRSRLSRRTPYSRDRLPPFETETRATILEDHPLLPECNTLTMHNVSYVRGLPCSVGFTLIQEWVVPWDMVLTREELVILRKCMHVCLCCANIDIMTSMMIHGYESWALHCHCSSPGSLQCIAGGQVLASWFRMVVDGAMFNQRFIWYREVVNYNMPKEVMFMSSVFMRGRHLIYLRLWYDGHVGSVVPAMSFGYSALHCGILNNIVVLCCSYCADLSEIRVRCCARRTRRLMLRAVRIIAEETTAMLYSCRTERRRQQFIRALLQHHRPILMHDYDSTPM